(19) United States
(12) Patent Application Publication
(10) Pub. No.: US 2014/0296176 A1
(43) Pub. Date: Oct. 2, 2014

(54) COMBINATION CANCER THERAPY WITH AN HSP90 INHIBITOR AND AN ANTIMETABOLITE

(71) Applicants: David Proia, Newton, MA (US); Julie Friedland, Burlington, MA (US)

(72) Inventors: David Proia, Newton, MA (US); Julie Friedland, Burlington, MA (US)

(73) Assignee: Synta Pharmaceuticals Corp., Lexington, MA (US)

(21) Appl. No.: 14/239,676

(22) PCT Filed: Aug. 17, 2012
(86) PCT No.: PCT/US2012/051316

§ 371 Date: Jun. 9, 2014

Related U.S. Application Data
(60) Provisional application No. 61/555,787, filed on Nov. 4, 2011.
(60) Provisional application No. 61/525,375, filed on Aug. 19, 2011.

(51) Int. Cl. A61K 31/675; A61K 31/7076; A61N 5/10; A61K 31/519; A61K 31/4196; A61K 31/7068; A61K 31/513

(52) U.S. Cl. CPC A61K 31/675; A61K 31/7068; A61K 31/7076; A61K 31/513; A61K 31/519; A61K 31/4196; A61N 5/10
USPC 514/45; 514/49; 514/274; 514/249; 514/265.1; 514/80; 600/1

(57) ABSTRACT

The invention provides a method of treating a subject with cancer, particularly leukemia, lymphoma, solid cancer such as colorectal cancer, gastric cancer, bladder cancer, non-small cell lung cancer, and breast cancer, comprising administering to the subject a compound of formulae (I) 40 or (Ia) in combination with an antimetabolite such as methotrexate, pemetrexed, cytarabine or nelarabine, or 5-fluorouracil, or capecitabine or their derivatives.

### CROSS-REFERENCE TO RELATED PATENTS

This application claims the benefit of priority to U.S. Provisional Patent Application Nos. 61/525,375, filed on Aug. 19, 2011, and 61/555,787, filed on Nov. 4, 2011. The contents of each of these applications are incorporated herein by reference in their entireties.

### BACKGROUND OF THE INVENTION

Although tremendous advances have been made in elucidating the genomic abnormalities that cause malignant cancer cells, currently available chemotherapy remains unsatisfactory, and the prognosis for the majority of patients diagnosed with cancer remains dismal. Most chemotherapeutic agents act on a specific molecular target thought to be involved in the development of the malignant phenotype. However, a complex network of signaling pathways regulate cell proliferation and the majority of malignant cancers are facilitated by multiple genetic abnormalities in these pathways. Therefore, it is less likely that a therapeutic agent that acts on one molecular target will be fully effective in curing a patient who has cancer.

Heat shock proteins (HSPs) are a class of chaperone proteins that are up-regulated in response to elevated temperature and other environmental stresses, such as ultraviolet light, nutrient deprivation and oxygen deprivation. HSPs act as chaperones to other cellular proteins (called client proteins), facilitate their proper folding and repair and aid in the refolding of misfolded client proteins. There are several known families of HSPs, each having its own set of client proteins. The Hsp90 family is one of the most abundant HSP families accounting for about 1-2% of proteins in a cell that is not under stress and increasing to about 4-6% in a cell under stress. Inhibition of Hsp90 results in the degradation of its client proteins via the ubiquitin proteasome pathway. Unlike other chaperone proteins, the client proteins of Hsp90 are mostly protein kinases or transcription factors involved in signal transduction, and a number of its client proteins have been shown to be involved in the progression of cancer.

### SUMMARY OF THE INVENTION

It is found that certain triazolone Hsp90 inhibitors and antimetabolite combinations are surprisingly effective at treating subjects with certain cancers without further increasing the side effect profile of the single agents. The particular combination therapies disclosed herein demonstrate surprising biological activity by demonstrating significant anticancer effects.

The combination therapy, in an embodiment, provides a method of treating a subject with cancer comprising administering to the subject an effective amount of an Hsp90 inhibitor according to formulae (I) or (Ia):

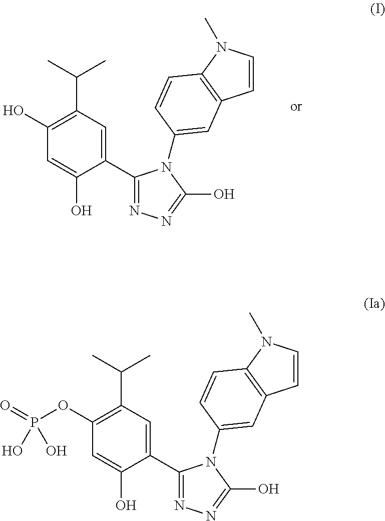 [SMILES: CC(C)c1cc(-c2nnc(O)n2-c2ccc3c(ccn3C)c2)c(O)cc1O ; CC(C)c1cc(-c2nnc(O)n2-c2ccc3c(ccn3C)c2)c(O)cc1OP(=O)(O)O]

or a pharmaceutically acceptable salt or a tautomer thereof, in combination with an antimetabolite such as methotrexate, pemetrexed, cytarabine (also called Ara-C), nelarabine (also called Ara-G), 5-fluorouracil, capecitabine or their derivatives.

In another embodiment, the method includes an Hsp90 inhibitor according to formulae (I) or (Ia) in combination with cytarabine. In an embodiment, the method includes an Hsp90 inhibitor according to formulae (I) or (Ia) in combination with nelarabine. In an embodiment, the method includes an Hsp90 inhibitor according to formulae (I) or (Ia) in combination with 5-fluorouracil. In an embodiment, the method includes an Hsp90 inhibitor according to formulae (I) or (Ia) in combination with capecitabine. In an embodiment, the method includes an Hsp90 inhibitor according to formulae (I) or (Ia) in combination with methotrexate. In an embodiment, the method includes an Hsp90 inhibitor according to formulae (I) or (Ia) in combination with pemetrexed.

In another embodiment, the method includes an Hsp90 inhibitor according to formula (I) in combination with cytarabine. In an embodiment, the method includes an Hsp90 inhibitor according to formula (I) in combination with nelarabine. In an embodiment, the method includes an Hsp90 inhibitor according to formula (I) in combination with 5-fluorouracil. In an embodiment, the method includes an Hsp90 inhibitor according to formula (I) in combination with capecitabine. In an embodiment, the method includes an Hsp90 inhibitor according to formula (I) in combination with methotrexate. In an embodiment, the method includes an Hsp90 inhibitor according to formula (I) in combination with pemetrexed.

In another embodiment, the method includes an Hsp90 inhibitor according to formulae (I) or (Ia) in combination with an antimetabolite for treating leukemia, lymphoma, solid tumor such as gastric cancer, colorectal cancer, bladder cancer, breast cancer, and non-small cell lung cancer. In another embodiment, the method includes an Hsp90 inhibitor according to formulae (I) or (Ia) in combination with cytarabine for treating leukemia or lymphoma or solid cancer. In an embodiment, the method includes an Hsp90 inhibitor according to formulae (I) or (Ia) in combination with nelarabine for treating leukemia or lymphoma or solid cancer. In an embodiment, the method includes an Hsp90 inhibitor according to formulae (I) or (Ia) in combination with 5-fluorouracil for treating leukemia or lymphoma or solid cancer. In an embodiment, the method includes an Hsp90 inhibitor according to formulae (I) or (Ia) in combination with capecitabine for treating leukemia or lymphoma or solid cancer. In an embodiment, the method includes an Hsp90 inhibitor according to formulae (I) or (Ia) in combination with methotrexate for treating leukemia or lymphoma or solid cancer. In an embodiment, the method includes an Hsp90 inhibitor according to formulae (I) or (Ia) in combination with pemetrexed for treating leukemia or lymphoma or solid cancer.

In an embodiment, the method includes an Hsp90 inhibitor according to formula (I) in combination with cytarabine for treating leukemia or lymphoma or solid cancer. In an embodiment, the method includes an Hsp90 inhibitor according to formula (I) in combination with nelarabine for treating leukemia or lymphoma or solid cancer. In an embodiment, the method includes an Hsp90 inhibitor according to formula (I) in combination with 5-fluorouracil for treating leukemia or lymphoma or solid cancer. In an embodiment, the method includes an Hsp90 inhibitor according to formula (I) in combination with capecitabine for treating leukemia or lymphoma or solid cancer. In an embodiment, the method includes an Hsp90 inhibitor according to formula (I) in combination with methotrexate for treating leukemia or lymphoma or solid cancer. In an embodiment, the method includes an Hsp90 inhibitor according to formula (I) in combination with pemetrexed for treating leukemia or lymphoma or solid cancer. In an embodiment, the leukemia is acute lymphoblastic leukemia (ALL). In an embodiment, the leukemia is chronic lymphocytic leukemia (CLL). In an embodiment, the leukemia is acute myelogenous leukemia (AML). In an embodiment, the leukemia is chronic myelogenous leukemia (CML). In an embodiment, the leukemia is T-cell acute lymphoblastic leukemia. In an embodiment, the leukemia is T cell prolymphocytic leukemia. In an embodiment, the lymphoma is non-Hodgkin's lymphoma. In an embodiment, the lymphoma is T-cell lymphoblastic lymphoma.

In an embodiment, the method includes an Hsp90 inhibitor according to formulae (I) or (Ia) in combination with cytarabine for the treatment of solid cancer such as colorectal cancer, bladder cancer, or gastric cancer. In an embodiment, the method includes an Hsp90 inhibitor according to formulae (I) or (Ia) in combination with nelarabine for the treatment of solid cancer such as colorectal cancer, bladder cancer, or gastric cancer. In an embodiment, the method includes an Hsp90 inhibitor according to formulae (I) or (Ia) in combination with 5-fluorouracil for the treatment of solid cancer such as colorectal cancer, bladder cancer, or gastric cancer. In an embodiment, the method includes an Hsp90 inhibitor according to formulae (I) or (Ia) in combination with capecitabine for the treatment of solid cancer such as colorectal cancer, bladder cancer, gastric cancer, non-small cell lung cancer, and breast cancer. In an embodiment, the method includes an Hsp90 inhibitor according to formulae (I) or (Ia) in combination with methotrexate for the treatment of solid cancer such as colorectal cancer, bladder cancer, gastric cancer, non-small cell lung cancer, and breast cancer. In an embodiment, the method includes an Hsp90 inhibitor according to formulae (I) or (Ia) in combination with pemetrexed for the treatment of solid cancer such as colorectal cancer, bladder cancer, gastric cancer, non-small cell lung cancer, and breast cancer. In an embodiment, the solid cancer is non-small cell lung cancer. In an embodiment, the non-small cell lung cancer has a KRAS mutation. In an embodiment, the method includes an Hsp90 inhibitor according to formulae (I) in combination with capecitabine for the treatment of colorectal cancer.

In another embodiment, the administration of the Hsp90 inhibitor and the antimetabolite are done concurrently. In an embodiment, the administration of the Hsp90 inhibitor and the antimetabolite are done sequentially. In an embodiment, the administration of the Hsp90 inhibitor and the antimetabolite are dosed independently. In an embodiment, the administration of the Hsp90 inhibitor and the antimetabolite are dosed separately. In an embodiment, the compound of formula (I) is administered intravenously once weekly at a dose of from about 100 mg/m² to about 200 mg/m². In an embodiment, the compound of formula (I) is administered intravenously twice weekly at a dose of from about 100 mg/m² to about 200 mg/m². In an embodiment, cytarabine is administered subcutaneously twice a day at a dose of from about 20 mg/m² to about 50 mg/m². In an embodiment, the compound of formula (I) is administered intravenously once weekly at an dose of from about 100 mg/m² to about 200 mg/m², and cytarabine is administered subcutaneously twice a day at a dose of from about 20 mg/m² to about 50 mg/m². In an embodiment, the compound of formula (I) is administered intravenously twice weekly at an dose of from about 100 mg/m² to about 200 mg/m², and cytarabine is administered subcutaneously twice a day at a dose of from about 20 mg/m² to about 50 mg/m². In an embodiment, nelarabine is administered intravenously three times or five times a week at a dose of from about 600 mg/m² to about 2000 mg/m². In an embodiment, the compound of formula (I) is administered intravenously twice weekly at an dose of from about 100 mg/m² to about 200 mg/m², and nelarabine is administered intravenously three times or five times a week at a dose of from about 600 mg/m² to about 2000 mg/m².

### BRIEF DESCRIPTION OF THE DRAWINGS

The foregoing and other objects, features and advantages of the combination therapies will be apparent from the following more particular description of some embodiments of the invention, as illustrated in the accompanying drawings in which like reference characters refer to the same parts throughout the different views. The drawings are not necessarily to scale, emphasis instead being placed upon illustrating the principles of the invention.

FIG. 1 shows dose viability response in MOLT-4 cells for ganetespib (compound (I) (top left), Ara-C (top right) and Ara-G (bottom left), respectively.

FIG. 2 shows substantially enhanced activity of nelarabine at lower concentrations in combination with ganetespib in treating MOLT-4 cells.

FIG. 3 shows substantially enhanced activity of cytarabine at lower concentrations in combination with ganetespib in treating MOLT-4 cells.

FIG. 4 shows significantly enhanced activity of ganetespib in combination with cytarabine or with nelarabine in treating MOLT-3 and Jurkat cells, respectively.

FIG. 5 is a graph showing dose dependence of ganetespib in HCT-116 cells with an IC₅₀ of approximately 32 nM.

FIG. 6 is a graph showing dose dependence of 5-fluorroural in HCT-116 cells with an IC₅₀ of about 4.5 μM.

FIG. 7 shows significant killing effect on HCT-116 cells by a combination of ganetespib at a concentration of 2 μM with 5-FU at a concentration of 2.3 W. Cells were exposed to ganetespib for 1 hour, washed and then treated with vehicle (DMSO) or fluorouracil for 3 days. Single agent chemotherapeutic was dosed for 3 days.

FIG. 8 further shows significant killing effect on HCT-116 cells by a combination of ganetespib at a concentration of 25 nM with 5-FU at a concentration of 3.4 W.

FIG. 9 shows potent activity in the form of IC₅₀ values of ganetespib in NSCLC cell lines with KRAS mutations after treatment with ganetespib for 72 hr.

FIG. 10 shows the effectiveness of combination of ganetespib with pemetrexed in treating NSCLC cell lines with various KRAS mutations for 72 hours in graphs representing ganetespib, pemetrexed, and a combination of the two, respectively.

FIG. 11 shows the effectiveness of combination of ganetespib with gemcitabine in treating NSCLC cell lines with various KRAS mutations for 72 hours in graphs representing ganetespib, gemcitabine, and a combination of the two, respectively.

### DETAILED DESCRIPTION OF THE INVENTION

### Definitions

Unless otherwise specified, the below terms used herein are defined as follows:

The articles “a”, “an” and “the” are used herein to refer to one or to more than one (i.e. to at least one) of the grammatical object of the article unless otherwise clearly indicated by contrast. By way of example, “an element” means one element or more than one element.

The term “including” is used herein to mean, and is used interchangeably with, the phrase “including but not limited to”.

The term “or” is used herein to mean, and is used interchangeably with, the term “and/or,” unless context clearly indicates otherwise.

The term “such as” is used herein to mean, and is used interchangeably, with the phrase “such as but not limited to”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

As used herein, the term “antimetabolite” refers to an antineoplastic drug that inhibits the utilization of a metabolite or a prodrug thereof. Examples of antimetabolites include methotrexate, pemetrexed, 5-fluorouracil, 5-fluorouracil prodrugs such as capecitabine, 5-fluorodeoxyuridine monophosphate, cytarabine, cytarabine prodrugs such as nelarabine, 5-azacytidine, gemcitabine, mercaptopurine, thioguanine, azathioprine, adenosine, pentostatin, erythrohydroxynonyladenine, and cladribine.

As used herein, the terms “subject”, “patient” and “mammal” are used interchangeably. The terms “subject” and “patient” refer to an animal (e.g., a bird such as a chicken, quail or turkey, or a mammal), preferably a mammal including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In another embodiment, the subject is a human.

As used herein, and unless otherwise indicated, the term “prodrug” means a derivative of a compound that can hydrolyze, oxidize, or otherwise react under biological conditions (in vitro or in vivo) to provide a compound described herein. Prodrugs may become active upon such reaction under biological conditions, or they may have activity in their unreacted forms. Examples of prodrugs contemplated herein include analogs or derivatives of compounds of formulae (I) or (Ia) or a compound in Tables 1 or 2 that comprise biohydrolyzable moieties such as biohydrolyzable amides, biohydrolyzable esters, biohydrolyzable carbamates, biohydrolyzable carbonates, biohydrolyzable ureides and phosphate analogues. Prodrugs can typically be prepared using well-known methods, such as those described by BURGER'S MEDICINAL CHEMISTRY AND DRUG DISCOVERY, (Manfred E. Wolff Ed., 5^{th} ed. (1995)) 172-178, 949-982.

As used herein, “Hsp90” includes each member of the family of heat shock proteins having a mass of about 90-kiloDaltons. For example, in humans the highly conserved Hsp90 family includes the cytosolic Hsp90α and Hsp90β isoforms, as well as GRP94, which is found in the endoplasmic reticulum, and HSP75/TRAP1, which is found in the mitochondrial matrix. As used herein, the terms of “Hsp90 inhibitor” or “Hsp90 inhibitory compound” refers to a compound that inhibits the activity of Hsp90 protein. Examples of Hsp90 inhibitors include triazolone compounds such as a compound of formulae (I) or (Ia), benzoquinone ansamycins such as geldanamycin and geldanamycin derivatives, and others such as IPI-493.

The terms “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features.

“Solid tumor,” as used herein, is understood as any pathogenic tumor that can be palpated or detected using imaging methods as an abnormal growth having three dimensions. A solid tumor is differentiated from a blood tumor such as leukemia. However, cells of a blood tumor are derived from bone marrow, therefore, the tissue producing the cancer cells is a solid tissue that can be hypoxic.

The KRAS oncogene is a critical gene in the development of a variety of cancers, and the mutation status of this gene is an important characteristic of many cancers. Mutation status of the gene can provide diagnostic, prognostic and predictive information for several cancers. The KRAS gene is a member of a family of genes (KRAS, NRAS and HRAS). KRAS is a member of the RAS family of oncogenes, a collection of small guanosine triphosphate (GTP)-binding proteins that integrate extracellular cues and activate intracellular signaling pathways to regulate cell proliferation, differentiation, and survival. Gain-of-function mutations that confer transforming capacity are frequently observed in KRAS, predominantly arising as single amino acid substitutions at amino acid residues G12, G13 or Q61. Constitutive activation of KRAS leads to the persistent stimulation of downstream signaling pathways that promote tumorigenesis, including the RAF/MEK/ERK and PI3K/AKT/mTOR cascades. In NSCLC, KRAS mutations are highly prevalent (20-30%) and are associated with unfavorable clinical outcomes. Mutations in KRAS appear mutually exclusive with those in EGFR in NSCLC tumors; more importantly, they can account for primary resistance to targeted EGFR TKI therapies. Mutations in the KRAS gene are common in many types of cancer, including pancreatic cancer (˜65%), colon cancer (˜40%), lung cancer (˜20%) and ovarian cancer (˜15%).

A variety of laboratory methods have been utilized to detect mutations in the KRAS gene. See, e.g., Jimeno et al, KRAS mutations and sensitivity to epidermal growth factor receptor inhibitors in colorectal cancer: practical application of patient selection. J. Clin. Oncol. 27, 1130-1135 (2009); Van Krieken et al, KRAS mutation testing for predicting response to anti-EGFR therapy for colorectal carcinoma: proposal for a European quality assurance program. Virchows Archiv. 453, 417-431 (2008). Most methods include the use of PCR to amplify the appropriate region of the KRAS gene, including exons 2 and 3, and then utilize different methods to distinguish wild-type from mutant sequences in key codons, such as 12 and 13. The detection methods include nucleic acid sequencing, allele-specific PCR methods, single-strand conformational polymorphism analysis, melt-curve analysis, probe hybridization and others. The main features for consideration for these molecular techniques are the ability to distinguish the appropriate spectrum of variants at the codons of interest and the sensitivity or limit of detection (LOD) for mutant alleles. Both of these parameters are important, given the fact that tumors may be very heterogeneous, both with regard to the percentage of tumor cells within a given tissue and the potential for genetic heterogeneity.

Moreover, many methods have also been developed for KRAS mutation analysis to address various specific issues, related to increased analytical sensitivity, and they include allele-specific PCR using amplification refractory mutation system (ARMS) technology or co-amplification at a lower denaturation temperature-PCR methods, pyrosequencing approaches and real-time PCR methods that use specific probe technologies, such as peptide nucleic acids. See, e.g., Pritchard et al, COLD-PCR enhanced melting curve analysis improves diagnostic accuracy for KRAS mutations in colorectal carcinoma. BMC Clin. Pathol. 10, 1-10 (2010); Weichart et al, KRAS genotyping of paraffin-embedded colorectal cancer tissue in routine diagnostics: comparison of methods and impact of histology. J. Mol. Diagn. 12, 35-42 (2010); Oliner et al, A comparability study of 5 commercial KRAS tests. Diagn. Pathol. 5, 23-29 (2010); Ogino et al, Brahmandan M et al. Sensitive sequencing method for KRAS mutation detection by pyrosequencing. J. Mol. Diagn. 4, 413-421 (2005).

There are several examples of laboratory-developed tests (LDTs) for detecting KRAS mutations, as well as a series of kits for research and for use in clinical diagnostics. For example, the TheraScreen® assay (DxS, Manchester, UK) is a CE-marked kit intended for the detection and qualitative assessment of seven somatic mutations in the KRAS gene, to aid clinicians in the identification of colorectal cancer patients who may benefit from anti-EGFR therapies, such as panitumumab and cetuximab. This assay uses an amplification refractory mutation system (ARMS), which is a version of allele-specific PCR; and detection of amplification products with Scorpion™ probes. See, e.g., TheraScreen® Package Insert, DsX, Manchester, UK (2009); Whitehall et al, A multicenter blinded study to evaluate KRAS mutation testing methodologies in the clinical setting. J. Mol. Diagn. 11, 543-552 (2009); Oliner et al, A comparability study of 5 commercial KRAS tests. Diagn. Pathol. 5, 23-29 (2010).

As used herein, the term “pharmaceutically acceptable salt” refers to a salt prepared from a compound of formulae (I) or (Ia), and a pharmaceutically acceptable inorganic or organic base. Suitable bases include hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like.

A pharmaceutically acceptable carrier may contain inert ingredients which do not unduly inhibit the biological activity of the compound(s) described herein. The pharmaceutically acceptable carriers should be biocompatible, i.e., non-toxic, non-inflammatory, non-immunogenic and devoid of other undesired reactions upon the administration to a subject. Standard pharmaceutical formulation techniques can be employed, such as those described in REMINGTON, J. P., REMINGTON'S PHARMACEUTICAL SCIENCES (Mack Pub. Co., 17^{th} ed., 1985). Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate, and the like. Methods for encapsulating compositions, such as in a coating of hard gelatin or cyclodextran, are known in the art. See BAKER, ET AL., CONTROLLED RELEASE OF BIOLOGICAL ACTIVE AGENTS, (John Wiley and Sons, 1986).

As used herein, the term “effective amount” refers to an amount of a compound described herein which is sufficient to reduce or ameliorate the severity, duration, progression, or onset of a disease or disorder, delay onset of a disease or disorder, retard or halt the advancement of a disease or disorder, cause the regression of a disease or disorder, prevent or delay the recurrence, development, onset or progression of a symptom associated with a disease or disorder, or enhance or improve the therapeutic effect(s) of another therapy. The precise amount of compound administered to a subject will depend on the mode of administration, the type and severity of the disease or condition and on the characteristics of the subject, such as general health, age, sex, body weight and tolerance to drugs. For example, for a proliferative disease or disorder, determination of an effective amount will also depend on the degree, severity and type of cell proliferation. The skilled artisan will be able to determine appropriate dosages depending on these and other factors. When co-administered with other therapeutic agents, e.g., when co-administered with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of the invention being used. In cases where no amount is expressly noted, an effective amount should be assumed. Non-limiting examples of an effective amount of a compound described herein are provided herein below.

In a specific embodiment, the invention provides a method of treating, managing, or ameliorating cancer, or one or more symptoms thereof, the method comprising administering to a subject in need thereof a dose of the Hsp90 inhibitor at least 150 μg/kg, at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds described herein once every day, once every 2 days, once every 3 days, once every 4 days, once every 5 days, once every 6 days, once every 7 days, once every 8 days, once every 10 days, once every two weeks, once every three weeks, or once a month.

As used herein, the terms “treat”, “treatment” and “treating” refer to the reduction or amelioration of the progression, severity and/or duration of a disease or disorder, delay of the onset of a disease or disorder, or the amelioration of one or more symptoms (preferably, one or more discernible symptoms) of a disease or disorder, resulting from the administration of one or more therapies (e.g., one or more therapeutic agents such as a compound of the invention). The terms “treat”, “treatment” and “treating” also encompass the reduction of the risk of developing a disease or disorder, and the delay or inhibition of the recurrence of a disease or disorder. In one embodiment, the disease or disorder being treated is cancer. In specific embodiments, the terms “treat”, “treatment” and “treating” refer to the amelioration of at least one measurable physical parameter of a disease or disorder, such as growth of a tumor, not necessarily discernible by the patient. In other embodiments the terms “treat”, “treatment” and “treating” refer to the inhibition of the progression of a disease or disorder, e.g., a proliferative disorder, either physically by the stabilization of a discernible symptom, physiologically by the stabilization of a physical parameter, or both. In another embodiment, the terms “treat”, “treatment” and “treating” of a proliferative disease or disorder refers to the reduction or stabilization of tumor size or cancerous cell count, and/or delay of tumor formation. In another embodiment, the terms “treat”, “treating” and “treatment” also encompass the administration of a compound described herein as a prophylactic measure to patients with a predisposition (genetic or environmental) to any disease or disorder described herein.

As used herein, the term “synergistic” refers to a combination of a compound described herein and another therapeutic agent, which, when taken together, is more effective than the additive effects of the individual therapies. A synergistic effect of a combination of therapies (e.g., a combination of therapeutic agents) permits the use of lower dosages of one or more of the therapeutic agent(s) and/or less frequent administration of the agent(s) to a subject with a disease or disorder, e.g., a proliferative disorder. The ability to utilize lower the dosage of one or more therapeutic agent and/or to administer the therapeutic agent less frequently reduces the toxicity associated with the administration of the agent to a subject without reducing the efficacy of the therapy in the treatment of a disease or disorder. In addition, a synergistic effect can result in improved efficacy of agents in the prevention, management or treatment of a disease or disorder, e.g. a proliferative disorder. Finally, a synergistic effect of a combination of therapies may avoid or reduce adverse or unwanted side effects associated with the use of either therapeutic agent alone.

As used herein, the term “in combination” refers to the use of more than one therapeutic agent. The use of the term “in combination” does not restrict the order in which the therapeutic agents are administered to a subject with a disease or disorder, e.g., a proliferative disorder. A first therapeutic agent, such as a compound described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent, such as an anti-cancer agent, to a subject with a disease or disorder, e.g. a proliferative disorder, such as cancer. In an embodiment, the Hsp90 inhibitor and the antimetabolite are dosed on independent schedules. In another embodiment, the Hsp90 inhibitor and the antimetabolite are dosed on approximately the same schedule. In another embodiment, the Hsp90 inhibitor and the antimetabolite are dosed concurrently or sequentially on the same day.

The recitation of an embodiment for a variable or aspect herein includes that embodiment as any single embodiment or in combination with any other embodiments or portions thereof.

The invention can be understood more fully by reference to the following detailed description and illustrative examples, which are intended to exemplify non-limiting embodiments of the invention.

The combination methods described herein utilize an Hsp90 inhibitor of formulae (I) or (Ia), or a pharmaceutically acceptable salt or a tautomer thereof,

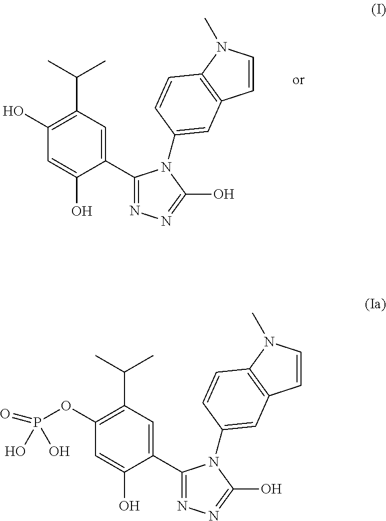 [SMILES: CC(C)c1cc(-c2nnc(O)n2-c2ccc3c(ccn3C)c2)c(O)cc1O ; CC(C)c1cc(-c2nnc(O)n2-c2ccc3c(ccn3C)c2)c(O)cc1OP(=O)(O)O]

for treating cancer, particularly, leukemia and lymphoma, or solid cancer such as gastric cancer, colorectal cancer, non-small cell lung cancer and bladder cancer, in combination with an antimetabolite such as methotrexate, pemetrexed, cytarabine, nelarabine, 5-fluorouracil, capecitabine, or their derivatives. In another embodiment, the combination may include one or more additional anticancer agents. In an embodiment, the one or more additional anticancer agents may include one or more of VEGF inhibitors such as bevacizumab, sunitinib, or sorafenib; one or more of EGFR inhibitors such as erlotinib, gefitinib or cetuximab; one or more of tyrosine kinase inhibitors such as imatinib; one or more of proteosome inhibitors such as bortezomib; one or more of taxanes such as paclitaxel and paclitaxel analogues; and one or more of ALK inhibitors such as crizotinib.

In an embodiment, the method includes an Hsp90 inhibitor according to formulae (I) or (Ia) in combination with cytarabine. In an embodiment, the method includes an Hsp90 inhibitor according to formulae (I) or (Ia) in combination with nelarabine. In an embodiment, the method includes an Hsp90 inhibitor according to formulae (I) or (Ia) in combination with 5-fluorouracil. In an embodiment, the method includes an Hsp90 inhibitor according to formulae (I) or (Ia) in combination with capecitabine. In an embodiment, the method includes an Hsp90 inhibitor according to formulae (I) or (Ia) in combination with methotrexate. In an embodiment, the method includes an Hsp90 inhibitor according to formulae (I) or (Ia) in combination with pemetrexed. In an embodiment, the method includes an Hsp90 inhibitor according to formula (I) in combination with cytarabine. In an embodiment, the method includes an Hsp90 inhibitor according to formula (I) in combination with nelarabine. In an embodiment, the method includes an Hsp90 inhibitor according to formula (I) in combination with 5-fluorouracil. In an embodiment, the method includes an Hsp90 inhibitor according to formula (I) in combination with capecitabine. In an embodiment, the method includes an Hsp90 inhibitor according to formulae (I) in combination with methotrexate. In an embodiment, the method includes an Hsp90 inhibitor according to formulae (I) in combination with pemetrexed.

In another embodiment, the method includes an Hsp90 inhibitor according to formulae (I) or (Ia) in combination with an antimetabolite for treating leukemia, lymphoma, solid tumor such as gastric cancer, colorectal cancer, bladder cancer, breast cancer, and non-small cell lung cancer. In an embodiment, the method includes an Hsp90 inhibitor according to formulae (I) or (Ia), in combination with cytarabine for treating leukemia or lymphoma or solid cancer. In an embodiment, the method includes an Hsp90 inhibitor according to formulae (I) or (Ia), in combination with nelarabine for treating leukemia or lymphoma or solid cancer. In an embodiment, the method includes an Hsp90 inhibitor according to formulae (I) or (Ia), in combination with 5-fluorouracil for treating leukemia or lymphoma or solid cancer. In an embodiment, the method includes an Hsp90 inhibitor according to formulae (I) or (Ia), in combination with capecitabine for treating leukemia or lymphoma or solid cancer. In an embodiment, the method includes an Hsp90 inhibitor according to formulae (I) or (Ia), in combination with methotrexate for treating leukemia or lymphoma or solid cancer. In an embodiment, the method includes an Hsp90 inhibitor according to formulae (I) or (Ia), in combination with pemetrexed for treating leukemia or lymphoma or solid cancer.

In another embodiment, the method includes an Hsp90 inhibitor according to formula (I), in combination with cytarabine for treating leukemia or lymphoma or solid cancer. In an embodiment, the method includes an Hsp90 inhibitor according to formula (I), in combination with nelarabine for treating leukemia or lymphoma or solid cancer. In an embodiment, the method includes an Hsp90 inhibitor according to formulae (I), in combination with 5-fluorouracil for treating leukemia or lymphoma or solid cancer. In an embodiment, the method includes an Hsp90 inhibitor according to formulae (I), in combination with capecitabine for treating leukemia or lymphoma or solid cancer. In an embodiment, the method includes an Hsp90 inhibitor according to formulae (I), in combination with methotrexate for treating leukemia or lymphoma or solid cancer. In an embodiment, the method includes an Hsp90 inhibitor according to formulae (I), in combination with pemetrexed for treating leukemia or lymphoma or solid cancer. In an embodiment, the leukemia is acute lymphoblastic leukemia (ALL). In an embodiment, the leukemia is chronic lymphocytic leukemia (CLL). In an embodiment, the leukemia is acute myelogenous leukemia (AML). In an embodiment, the leukemia is chronic myelogenous leukemia (CML). In an embodiment, the leukemia is T-cell acute lymphoblastic leukemia. In an embodiment, the leukemia is T cell prolymphocytic leukemia. In an embodiment, the lymphoma is non-Hodgkin's lymphoma. In an embodiment, the lymphoma is T-cell lymphoblastic lymphoma.

In an embodiment, the method includes an Hsp90 inhibitor according to formulae (I) or (Ia) in combination with cytarabine for treating solid cancer such as colorectal cancer, bladder cancer, breast cancer, non-small cell lung cancer, and gastric cancer. In an embodiment, the method includes an Hsp90 inhibitor according to formulae (I) or (Ia) in combination with nelarabine for treating solid cancer such as colorectal cancer, bladder cancer, breast cancer, non-small cell lung cancer, and gastric cancer. In an embodiment, the method includes an Hsp90 inhibitor according to formulae (I) or (Ia) in combination with 5-fluorouracil for treating solid cancer such as colorectal cancer, bladder cancer, breast cancer, non-small cell lung cancer, and gastric cancer. In an embodiment, the method includes an Hsp90 inhibitor according to formulae (I) or (Ia) in combination with capecitabine for treating solid cancer such as colorectal cancer, bladder cancer, breast cancer, non-small cell lung cancer, and gastric cancer. In an embodiment, the method includes an Hsp90 inhibitor according to formulae (I) or (Ia) in combination with methotrexate for treating solid cancer such as colorectal cancer, bladder cancer, breast cancer, non-small cell lung cancer, and gastric cancer. In an embodiment, the method includes an Hsp90 inhibitor according to formulae (I) or (Ia) in combination with pemetrexed for treating solid cancer such as colorectal cancer, bladder cancer, breast cancer, non-small cell lung cancer, and gastric cancer. In an embodiment, the solid cancer is non-small cell lung cancer. In an embodiment, the non-small cell lung cancer has a mutation in KRAS. In an embodiment, the method includes an Hsp90 inhibitor according to formulae (I) or (Ia) in combination with capecitabine for treating colorectal cancer.

The method includes administering to a subject in need thereof an effective amount of an Hsp90 inhibitory compound according to formulae (I) or (Ia) and an antimetabolite such as methotrexate, pemetrexed, cytarabine, nelarabine, 5-fluorouracil, capecitabine, or their derivatives. In an embodiment, the administration of the Hsp90 inhibitor and the antimetabolite are done concurrently. In another embodiment, the administration of the Hsp90 inhibitor and the antimetabolite are done sequentially. In another embodiment, the administration of the Hsp90 inhibitor and the antimetabolite are dosed independently. In another embodiment, the administration of the Hsp90 inhibitor and the antimetabolite are dosed separately. In another embodiment, the administration of the Hsp90 inhibitor and the antimetabolite are done until the cancer is cured or stabilized or improved.

In an embodiment, the compound of formula (I) is administered intravenously once weekly at a dose of from about 100 mg/m² to about 200 mg/m². In an embodiment, the compound of formula (I) is administered intravenously once weekly at a dose of about 150 mg/m². In an embodiment, the compound of formula (I) is administered intravenously once weekly at a dose of about 175 mg/m². In an embodiment, the compound of formula (I) is administered intravenously once weekly at a dose of about 200 mg/m². In an embodiment, the compound of formula (I) is administered intravenously twice weekly at a dose of from about 100 mg/m² to about 200 mg/m². In an embodiment, cytarabine is administered subcutaneously twice a day at a dose of from about 20 mg/m² to about 50 mg/m². In an embodiment, the compound of formula (I) is administered intravenously once weekly at an dose of from about 100 mg/m² to about 200 mg/m², and cytarabine is administered subcutaneously twice a day at a dose of from about 20 mg/m² to about 50 mg/m². In an embodiment, the compound of formula (I) is administered intravenously twice weekly at an dose of from about 100 mg/m² to about 200 mg/m², and cytarabine is administered subcutaneously twice a day at a dose of from about 20 mg/m² to about 50 mg/m².

In an embodiment, the compound of formula (I) is administered intravenously once weekly at a dose of from about 100 mg/m² to about 200 mg/m². In an embodiment, the compound of formula (I) is administered intravenously once weekly at a dose of about 150 mg/m². In an embodiment, the compound of formula (I) is administered intravenously once weekly at a dose of about 175 mg/m². In an embodiment, the compound of formula (I) is administered intravenously once weekly at a dose of about 200 mg/m². In an embodiment, the compound of formula (I) is administered intravenously twice weekly at a dose of from about 100 mg/m² to about 200 mg/m². In an embodiment, nelarabine is administered intravenously three times or five times a week at a dose of from about 600 mg/m² to about 2000 mg/m². In an embodiment, nelarabine is administered at a dose of about 650 mg/m² five times a week. In an embodiment, nelarabine is administered at a dose of about 1500 mg/m² three times a week. In an embodiment, the compound of formula (I) is administered intravenously once weekly at an dose of from about 100 mg/m² to about 200 mg/m², and nelarabine is administered intravenously three times or five times a week at a dose of from about 600 mg/m² to about 2000 mg/m². In an embodiment, the compound of formula (I) is administered intravenously twice weekly at an dose of from about 100 mg/m² to about 200 mg/m², and nelarabine is administered intravenously three times or five times a week at a dose of from about 600 mg/m² to about 2000 mg/m².

In an embodiment, the compound of formula (I) is administered intravenously once weekly at a dose of from about 100 mg/m² to about 200 mg/m². In an embodiment, the compound of formula (I) is administered intravenously once weekly at a dose of about 150 mg/m². In an embodiment, the compound of formula (I) is administered intravenously once weekly at a dose of about 175 mg/m². In an embodiment, the compound of formula (I) is administered intravenously once weekly at a dose of about 200 mg/m². In an embodiment, the compound of formula (I) is administered intravenously twice weekly at a dose of from about 100 mg/m² to about 200 mg/m². In an embodiment, capecitabine is administered at a dose from about 200 mg/m² to about 3000 mg/m². In an embodiment, capecitabine is administered at about 1250 mg/m². In an embodiment, capecitabine is administered orally at about 1250 mg/m² twice daily. In an embodiment, capecitabine is administered orally at about 1250 mg/m² twice daily for two weeks followed by one week rest. In an embodiment, the combination treatment further comprises radiotherapy.

In an embodiment, the combination therapy includes a pharmaceutical composition or a single unit dosage form containing both an Hsp90 inhibitor and an antimetabolite. Pharmaceutical combinations and dosage forms described herein comprise the two active ingredients in relative amounts and formulated in such a way that a given pharmaceutical combination or dosage form can be used to treat cancer. Preferred pharmaceutical combinations and dosage forms comprise a compound of formulae (I) or (Ia), or a tautomer or a pharmaceutically acceptable salt thereof, in combination with an antimetabolite. In other embodiments, the Hsp90 inhibitor and the antimetabolite may be in individual or separate pharmaceutical compositions, depending on the dosing schedules, preferred routes of administration, and available formulations of the two inhibitors. Optionally, these embodiments can also contain one or more additional therapeutic agents.

The pharmaceutical combinations described herein are formulated to be compatible with its intended route of administration. Examples of routes of administration include parenteral, e.g., intravenous, intradermal, subcutaneous, oral, intranasal (e.g., inhalation), transdermal (topical), transmucosal, and rectal administration. In a specific embodiment, the combination is formulated in accordance with routine procedures as a pharmaceutical composition adapted for intravenous, subcutaneous, intramuscular, oral, intranasal or topical administration to human beings. In an embodiment, the combination is formulated in accordance with routine procedures for subcutaneous administration to human beings.

The Hsp90 inhibitory compound of formulae (I) or (Ia) described herein can be also formulated into or administered by controlled release means or by delivery devices that are well known to those of ordinary skill in the art. Examples include those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566.

Some of the disclosed methods can be also effective at treating subjects whose cancer has become “drug resistant” or “multi-drug resistant”. A cancer which initially responded to an anti-cancer drug becomes resistant to the anti-cancer drug when the anti-cancer drug is no longer effective in treating the subject with the cancer. For example, many tumors will initially respond to treatment with an anti-cancer drug by decreasing in size or even going into remission, only to develop resistance to the drug. “Drug resistant” tumors are characterized by a resumption of their growth and/or reappearance after having seemingly gone into remission, despite the administration of increased dosages of the anti-cancer drug. Cancers that have developed resistance to two or more anti-cancer drugs are said to be “multi-drug resistant”. For example, it is common for cancers to become resistant to three or more anti-cancer agents, often five or more anti-cancer agents and at times ten or more anti-cancer agents.

In another embodiment, the present method includes treating, managing, or ameliorating cancer, or one or more symptoms thereof, comprising administering to a subject in need thereof a triazolone compound according to formulae (I) or (Ia), in combination with an antimetabolite such as methotrexate, pemetrexed, cytarabine, or nelarabine, or 5-fluorouracil or capecitabine, or their derivatives, wherein the cancer is leukemia, acute lymphoblastic leukemia (ALL), chronic lymphocytic leukemia (CLL), acute myelogenous leukemia (AML), chronic myelogenous leukemia (CML), T-cell acute lymphoblastic leukemia, T cell prolymphocytic leukemia, lymphoma, non-Hodgkin's lymphoma, T-cell lymphoblastic lymphoma, or solid cancer such as gastric cancer, colorectal cancer, bladder cancer, breast cancer, and non-small cell lung cancer. In an embodiment, the non-small cell lung cancer has a KRAS mutation.

In an embodiment, the amount of the compound of formulae (I) or (Ia) administered is from about 2 mg/m² to about 500 mg/m², for example, from about 100 mg/m² to about 500 mg/m², from about 125 mg/m² to about 500 mg/m², from about 150 mg/m² to about 500 mg/m² or from about 175 mg/m² to about 500 mg/m². In an embodiment, the amount of the compound of formula (I) or (Ia) administered is about 100 mg/m² to about 300 mg/m², from about 125 mg/m² to about 300 mg/m², from about 150 mg/m² to about 300 mg/m² or from about 175 mg/m² to about 300 mg/m². In some embodiments, the amount of the compound of formula (I) or (Ia) administered is about 2 mg/m², 4 mg/m², about 7 mg/m², about 10 mg/m², about 14 mg/m², about 19 mg/m², about 23 mg/m², about 25 mg/m², about 33 mg/m², about 35 mg/m², about 40 mg/m², about 48 mg/m², about 49 mg/m², about 50 mg/m², about 65 mg/m², about 75 mg/m², about 86 mg/m², about 100 mg/m², about 110 mg/m², about 114 mg/m², about 120 mg/m², about 144 mg/m², about 150 mg/m², about 173 mg/m², about 180 mg/m², about 200 mg/m², about 216 mg/m² or about 259 mg/m².

In an embodiment, the administration of the compound of formula (I) or (Ia) can be once weekly, twice weekly. The language “twice weekly” includes administration of the compound of formula (I) or (Ia) two times in about 7 days. For example, the first dose of the compound of formula (I) or (Ia) is administered on day 1, and the second dose of the compound of formula (I) or (Ia) may be administered on day 2, day 3, day 4, day 5, day 6 or day 7. In some embodiments, the twice weekly administration occurs on days 1 and 3 or days 1 and 4.

In another embodiment, the method of treating a subject with cancer includes administering to the subject an effective amount of the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with an effective amount of an antimetabolite such as methotrexate, pemetrexed, cytarabine or nelarabine or 5-fluorouracil, capecitabine, or their derivatives.

In another embodiment, the method of treating a subject with cancer includes administering to the subject an effective amount of the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with an effective amount of cytarabine.

In another embodiment, the method of treating a subject with cancer includes administering to the subject an effective amount of the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with an effective amount of nelarabine.

In another embodiment, the method of treating a subject with cancer includes administering to the subject an effective amount of the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with an effective amount of 5-fluorouracil.

In another embodiment, the method of treating a subject with cancer includes administering to the subject an effective amount of the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with an effective amount of capecitabine. In an embodiment, the combination treatment is further combined with radiotherapy.

In another embodiment, the method of treating a subject with cancer includes administering to the subject an effective amount of the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with an effective amount of 5-methotrexatel.

In another embodiment, the method of treating a subject with cancer includes administering to the subject an effective amount of the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with an effective amount of 5-pemetrexed.

In another embodiment, the method of treating a subject with cancer includes administering to the subject an effective amount of the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with an effective amount of cytarabine.

In another embodiment, the method of treating a subject with cancer includes administering to the subject an effective amount of the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with an effective amount of nelarabine.

In another embodiment, the method of treating a subject with cancer includes administering to the subject an effective amount of the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with an effective amount of 5-fluorouracil.

In another embodiment, the method of treating a subject with cancer includes administering to the subject an effective amount of the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with an effective amount of capecitabine. In an embodiment, the combination treatment is further combined with radiotherapy.

In another embodiment, the method of treating a subject with cancer includes administering to the subject an effective amount of the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with an effective amount of methotrexate.

In another embodiment, the method of treating a subject with cancer includes administering to the subject an effective amount of the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with an effective amount of pemetrexed.

In another embodiment, the method of treating a subject with cancer includes administering to the subject an effective amount of the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with cytarabine, wherein the cancer is leukemia, acute lymphoblastic leukemia (ALL), chronic lymphocytic leukemia (CLL), acute myelogenous leukemia (AML), chronic myelogenous leukemia (CML), T-cell acute lymphoblastic leukemia, T cell prolymphocytic leukemia, lymphoma, non-Hodgkin's lymphoma, T-cell lymphoblastic lymphoma, or solid cancer such as gastric cancer, colorectal cancer, bladder cancer, breast cancer, and non-small cell lung cancer. In an embodiment, the non-small cell lung cancer has a KRAS mutation.

In another embodiment, the method of treating a subject with cancer includes administering to the subject an effective amount of the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with nelarabine, wherein the cancer is leukemia, acute lymphoblastic leukemia (ALL), chronic lymphocytic leukemia (CLL), acute myelogenous leukemia (AML), chronic myelogenous leukemia (CML), T-cell acute lymphoblastic leukemia, T cell prolymphocytic leukemia, lymphoma, non-Hodgkin's lymphoma, T-cell lymphoblastic lymphoma or solid cancer such as gastric cancer, colorectal cancer, bladder cancer, breast cancer, and non-small cell lung cancer. In an embodiment, the non-small cell lung cancer has a KRAS mutation.

In another embodiment, the method of treating a subject with cancer includes administering to the subject an effective amount of the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with 5-fluorouracil, wherein the cancer is leukemia, acute lymphoblastic leukemia (ALL), chronic lymphocytic leukemia (CLL), acute myelogenous leukemia (AML), chronic myelogenous leukemia (CML), T-cell acute lymphoblastic leukemia, T cell prolymphocytic leukemia, lymphoma, non-Hodgkin's lymphoma, T-cell lymphoblastic lymphoma, or solid cancer such as gastric cancer, colorectal cancer, bladder cancer, breast cancer, and non-small cell lung cancer. In an embodiment, the non-small cell lung cancer has a KRAS mutation.

In another embodiment, the method of treating a subject with cancer includes administering to the subject an effective amount of the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with capecitabine, wherein the cancer is leukemia, acute lymphoblastic leukemia (ALL), chronic lymphocytic leukemia (CLL), acute myelogenous leukemia (AML), chronic myelogenous leukemia (CML), T-cell acute lymphoblastic leukemia, T cell prolymphocytic leukemia, lymphoma, non-Hodgkin's lymphoma, T-cell lymphoblastic lymphoma, or solid cancer such as gastric cancer, colorectal cancer, bladder cancer, breast cancer, and non-small cell lung cancer. In an embodiment, the non-small cell lung cancer has a KRAS mutation. In an embodiment, the combination treatment is further combined with radiotherapy.

In another embodiment, the method of treating a subject with cancer includes administering to the subject an effective amount of the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with methotrexate, wherein the cancer is leukemia, acute lymphoblastic leukemia (ALL), chronic lymphocytic leukemia (CLL), acute myelogenous leukemia (AML), chronic myelogenous leukemia (CML), T-cell acute lymphoblastic leukemia, T cell prolymphocytic leukemia, lymphoma, non-Hodgkin's lymphoma, T-cell lymphoblastic lymphoma, or solid cancer such as gastric cancer, colorectal cancer, bladder cancer, breast cancer, and non-small cell lung cancer. In an embodiment, the non-small cell lung cancer has a KRAS mutation. In an embodiment, the combination treatment is further combined with radiotherapy.

In another embodiment, the method of treating a subject with cancer includes administering to the subject an effective amount of the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with pemetrexed, wherein the cancer is leukemia, acute lymphoblastic leukemia (ALL), chronic lymphocytic leukemia (CLL), acute myelogenous leukemia (AML), chronic myelogenous leukemia (CML), T-cell acute lymphoblastic leukemia, T cell prolymphocytic leukemia, lymphoma, non-Hodgkin's lymphoma, T-cell lymphoblastic lymphoma, or solid cancer such as gastric cancer, colorectal cancer, bladder cancer, breast cancer, and non-small cell lung cancer. In an embodiment, the non-small cell lung cancer has a KRAS mutation. In an embodiment, the combination treatment is further combined with radiotherapy.

In another embodiment, the method of treating a subject with cancer includes administering to the subject an effective amount of the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with cytarabine, wherein the cancer is leukemia, acute lymphoblastic leukemia (ALL), chronic lymphocytic leukemia (CLL), acute myelogenous leukemia (AML), chronic myelogenous leukemia (CML), T-cell acute lymphoblastic leukemia, T cell prolymphocytic leukemia, lymphoma, non-Hodgkin's lymphoma, T-cell lymphoblastic lymphoma, or solid cancer such as gastric cancer, colorectal cancer, bladder cancer, breast cancer, and non-small cell lung cancer. In an embodiment, the non-small cell lung cancer has a KRAS mutation.

In another embodiment, the method of treating a subject with cancer includes administering to the subject an effective amount of the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with nelarabine, wherein the cancer is leukemia, acute lymphoblastic leukemia (ALL), chronic lymphocytic leukemia (CLL), acute myelogenous leukemia (AML), chronic myelogenous leukemia (CML), T-cell acute lymphoblastic leukemia, T cell prolymphocytic leukemia, lymphoma, non-Hodgkin's lymphoma, T-cell lymphoblastic lymphoma, or solid cancer such as gastric cancer, colorectal cancer, bladder cancer, breast cancer, and non-small cell lung cancer. In an embodiment, the non-small cell lung cancer has a KRAS mutation.

In another embodiment, the method of treating a subject with cancer includes administering to the subject an effective amount of the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with 5-fluorouracil, wherein the cancer is leukemia, acute lymphoblastic leukemia (ALL), chronic lymphocytic leukemia (CLL), acute myelogenous leukemia (AML), chronic myelogenous leukemia (CML), T-cell acute lymphoblastic leukemia, T cell prolymphocytic leukemia, lymphoma, non-Hodgkin's lymphoma, T-cell lymphoblastic lymphoma, or solid cancer such as gastric cancer, colorectal cancer, bladder cancer, breast cancer, and non-small cell lung cancer. In an embodiment, the non-small cell lung cancer has a KRAS mutation.

In another embodiment, the method of treating a subject with cancer includes administering to the subject an effective amount of the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with capecitabine, wherein the cancer is leukemia, acute lymphoblastic leukemia (ALL), chronic lymphocytic leukemia (CLL), acute myelogenous leukemia (AML), chronic myelogenous leukemia (CML), T-cell acute lymphoblastic leukemia, T cell prolymphocytic leukemia, lymphoma, non-Hodgkin's lymphoma, T-cell lymphoblastic lymphoma, or solid cancer such as gastric cancer, colorectal cancer, bladder cancer, breast cancer, and non-small cell lung cancer. In an embodiment, the non-small cell lung cancer has a KRAS mutation.

In another embodiment, the method of treating a subject with cancer includes administering to the subject an effective amount of the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with methotrexate, wherein the cancer is leukemia, acute lymphoblastic leukemia (ALL), chronic lymphocytic leukemia (CLL), acute myelogenous leukemia (AML), chronic myelogenous leukemia (CML), T-cell acute lymphoblastic leukemia, T cell prolymphocytic leukemia, lymphoma, non-Hodgkin's lymphoma, T-cell lymphoblastic lymphoma, or solid cancer such as gastric cancer, colorectal cancer, bladder cancer, breast cancer, and non-small cell lung cancer. In an embodiment, the non-small cell lung cancer has a KRAS mutation.

In another embodiment, the method of treating a subject with cancer includes administering to the subject an effective amount of the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with pemetrexed, wherein the cancer is leukemia, acute lymphoblastic leukemia (ALL), chronic lymphocytic leukemia (CLL), acute myelogenous leukemia (AML), chronic myelogenous leukemia (CML), T-cell acute lymphoblastic leukemia, T cell prolymphocytic leukemia, lymphoma, non-Hodgkin's lymphoma, T-cell lymphoblastic lymphoma, or solid cancer such as gastric cancer, colorectal cancer, bladder cancer, breast cancer, and non-small cell lung cancer. In an embodiment, the non-small cell lung cancer has a KRAS mutation.

In yet another embodiment, the method of treating a subject with cancer, wherein the subject is being or has been treated with a chemotherapeutic agent, includes administering to the subject an effective amount of the triazolone compound according to formulae (I) or (Ia), in combination with an antimetabolite such as methotrexate, pemetrexed, cytarabine, or nelarabine, or 5-fluorouracil, or capecitabine, or their derivatives.

In an embodiment, the method of treating a subject with cancer, wherein the subject is being or has been treated with a chemotherapeutic agent, includes administering to the subject an effective amount of the triazolone compound according to formulae (I) or (Ia), in combination with an antimetabolite such as methotrexate, pemetrexed, cytarabine or nelarabine or 5-fluorouracol or capecitabine, wherein the cancer is leukemia, acute lymphoblastic leukemia (ALL), chronic lymphocytic leukemia (CLL), acute myelogenous leukemia (AML), chronic myelogenous leukemia (CML), T-cell acute lymphoblastic leukemia, T cell prolymphocytic leukemia, lymphoma, non-Hodgkin's lymphoma, T-cell lymphoblastic lymphoma, or solid cancer such as gastric cancer, colorectal cancer, bladder cancer, breast cancer, and non-small cell lung cancer. In an embodiment, the non-small cell lung cancer has a KRAS mutation.

In another embodiment, the method of treating a subject with cancer, wherein the subject is being or has been treated with a chemotherapeutic agent, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with an antimetabolite such as methotrexate, pemetrexed, cytarabine, or nelarabine, or 5-fluorouracil, or capecitabine, or their derivatives.

In another embodiment, the method of treating a subject with cancer, wherein the subject is being or has been treated with a chemotherapeutic agent, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with cytarabine.

In another embodiment, the method of treating a subject with cancer, wherein the subject is being or has been treated with a chemotherapeutic agent, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with nelarabine.

In another embodiment, the method of treating a subject with cancer, wherein the subject is being or has been treated with a chemotherapeutic agent, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with 5-fluorouracil.

In another embodiment, the method of treating a subject with cancer, wherein the subject is being or has been treated with a chemotherapeutic agent, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with capecitabine.

In another embodiment, the method of treating a subject with cancer, wherein the subject is being or has been treated with a chemotherapeutic agent, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with methotrexate.

In another embodiment, the method of treating a subject with cancer, wherein the subject is being or has been treated with a chemotherapeutic agent, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with pemetrexed.

In another embodiment, the method of treating a subject with cancer, wherein the subject is being or has been treated with a chemotherapeutic agent, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with an antimetabolite such as methotrexate, pemetrexed, cytarabine, or nelarabine, or 5-fluorouracil, or capecitabine, or their derivatives.

In another embodiment, the method of treating a subject with cancer, wherein the subject is being or has been treated with a chemotherapeutic agent, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with cytarabine.

In another embodiment, the method of treating a subject with cancer, wherein the subject is being or has been treated with a chemotherapeutic agent, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with nelarabine.

In another embodiment, the method of treating a subject with cancer, wherein the subject is being or has been treated with a chemotherapeutic agent, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with 5-fluorouracil.

In another embodiment, the method of treating a subject with cancer, wherein the subject is being or has been treated with a chemotherapeutic agent, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with capecitabine.

In another embodiment, the method of treating a subject with cancer, wherein the subject is being or has been treated with a chemotherapeutic agent, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with methotrexate.

In another embodiment, the method of treating a subject with cancer, wherein the subject is being or has been treated with a chemotherapeutic agent, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with pemetrexed.

In an embodiment, the method of treating a subject with cancer, wherein the subject is being or has been treated with a chemotherapeutic agent, includes administering to the subject an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with an antimetabolite such as methotrexate, pemetrexed, cytarabine or nelarabine, wherein the cancer is leukemia, acute lymphoblastic leukemia (ALL), chronic lymphocytic leukemia (CLL), acute myelogenous leukemia (AML), chronic myelogenous leukemia (CML), T-cell acute lymphoblastic leukemia, T cell prolymphocytic leukemia, lymphoma, non-Hodgkin's lymphoma, T-cell lymphoblastic lymphoma, or solid cancer such as gastric cancer, colorectal cancer, bladder cancer, breast cancer, and non-small cell lung cancer. In an embodiment, the non-small cell lung cancer has a KRAS mutation.

In an embodiment, the method of treating a subject with cancer, wherein the subject is being or has been treated with a chemotherapeutic agent, includes administering to the subject an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with an antimetabolite such as methotrexate, pemetrexed, cytarabine or nelarabine, wherein the cancer is leukemia, acute lymphoblastic leukemia (ALL), chronic lymphocytic leukemia (CLL), acute myelogenous leukemia (AML), chronic myelogenous leukemia (CML), T-cell acute lymphoblastic leukemia, T cell prolymphocytic leukemia, lymphoma, non-Hodgkin's lymphoma, T-cell lymphoblastic lymphoma, or solid cancer such as gastric cancer, colorectal cancer, bladder cancer, breast cancer, and non-small cell lung cancer. In an embodiment, the non-small cell lung cancer has a KRAS mutation.

In an embodiment, the method of treating a subject with cancer, wherein the subject has proven refractory to other therapies but is no longer on these therapies, includes administering to the subject an effective amount of the triazolone compound according to formulae (I) or (Ia), in combination with an antimetabolite such as methotrexate, pemetrexed, cytarabine or nelarabine or 5-fluorouracil or capecitabine, wherein the cancer is leukemia, acute lymphoblastic leukemia (ALL), chronic lymphocytic leukemia (CLL), acute myelogenous leukemia (AML), chronic myelogenous leukemia (CML), T-cell acute lymphoblastic leukemia, T cell prolymphocytic leukemia, lymphoma, non-Hodgkin's lymphoma, T-cell lymphoblastic lymphoma, or solid cancer such as gastric cancer, colorectal cancer, bladder cancer, breast cancer, and non-small cell lung cancer. In an embodiment, the non-small cell lung cancer has a KRAS mutation.

In another embodiment, the method of treating a subject with cancer, wherein the subject has proven refractory to other therapies but is no longer on these therapies, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with an antimetabolite such as methotrexate, pemetrexed, cytarabine, or nelarabine, or 5-fluorouracil, or capecitabine, or their derivatives.

In another embodiment, the method of treating a subject with cancer, wherein the subject has proven refractory to other therapies but is no longer on these therapies, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with cytarabine.

In another embodiment, the method of treating a subject with cancer, wherein the subject has proven refractory to other therapies but is no longer on these therapies, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with nelarabine.

In another embodiment, the method of treating a subject with cancer, wherein the subject has proven refractory to other therapies but is no longer on these therapies, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with 5-fluorouracil.

In another embodiment, the method of treating a subject with cancer, wherein the subject has proven refractory to other therapies but is no longer on these therapies, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with capecitabine.

In another embodiment, the method of treating a subject with cancer, wherein the subject has proven refractory to other therapies but is no longer on these therapies, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with methotrexate.

In another embodiment, the method of treating a subject with cancer, wherein the subject has proven refractory to other therapies but is no longer on these therapies, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with pemetrexed.

In another embodiment, the method of treating a subject with cancer, wherein the subject has proven refractory to other therapies but is no longer on these therapies, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with an antimetabolite such as methotrexate, pemetrexed, cytarabine or nelarabine or 5-fluorouracil or capecitabine or their derivatives.

In another embodiment, the method of treating a subject with cancer, wherein the subject has proven refractory to other therapies but is no longer on these therapies, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with cytarabine.

In another embodiment, the method of treating a subject with cancer, wherein the subject has proven refractory to other therapies but is no longer on these therapies, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with nelarabine.

In another embodiment, the method of treating a subject with cancer, wherein the subject has proven refractory to other therapies but is no longer on these therapies, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with 5-fluorouracil.

In another embodiment, the method of treating a subject with cancer, wherein the subject has proven refractory to other therapies but is no longer on these therapies, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with capecitabine.

In another embodiment, the method of treating a subject with cancer, wherein the subject has proven refractory to other therapies but is no longer on these therapies, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with methotrexate.

In another embodiment, the method of treating a subject with cancer, wherein the subject has proven refractory to other therapies but is no longer on these therapies, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with pemetrexed.

In an embodiment, the method of treating a subject with cancer, wherein the subject has proven refractory to other therapies but is no longer on these therapies, includes administering to the subject an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with an antimetabolite such as methotrexate, pemetrexed, cytarabine or nelarabine, wherein the cancer is leukemia, acute lymphoblastic leukemia (ALL), chronic lymphocytic leukemia (CLL), acute myelogenous leukemia (AML), chronic myelogenous leukemia (CML), T-cell acute lymphoblastic leukemia, T cell prolymphocytic leukemia, lymphoma, non-Hodgkin's lymphoma, T-cell lymphoblastic lymphoma, or solid cancer such as gastric cancer, colorectal cancer, bladder cancer, breast cancer, and non-small cell lung cancer. In an embodiment, the non-small cell lung cancer has a KRAS mutation.

In an embodiment, the method of treating a subject with cancer, wherein the subject has proven refractory to other therapies but is no longer on these therapies, includes administering to the subject an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with an antimetabolite such as methotrexate, pemetrexed, cytarabine or nelarabine, wherein the cancer is leukemia, acute lymphoblastic leukemia (ALL), chronic lymphocytic leukemia (CLL), acute myelogenous leukemia (AML), chronic myelogenous leukemia (CML), T-cell acute lymphoblastic leukemia, T cell prolymphocytic leukemia, lymphoma, non-Hodgkin's lymphoma, T-cell lymphoblastic lymphoma, or solid cancer such as gastric cancer, colorectal cancer, bladder cancer, breast cancer, and non-small cell lung cancer. In an embodiment, the non-small cell lung cancer has a KRAS mutation.

In another embodiment, the method includes inhibiting the growth of a cancer or tumor cell comprising the steps of: (a) contacting the cell with an effective amount of a compound of formulae (I) or (Ia), or tautomer or a pharmaceutically acceptable salt thereof; and (b) exposing the cell to an effective amount of an antimetabolite such as methotrexate, pemetrexed, cytarabine, or nelarabine, or 5-fluorouracil, or capecitabine, or their derivatives.

In another embodiment, the method includes inhibiting the growth of a cancer or tumor cell comprising the steps of: (a) contacting the cell with an effective amount of a compound of -(2,4-dihydroxy-5-isopropyl-phenyl)-4-(1-methyl-indol-5-yl)-5-hydroxy-[1,2,4]triazole, or a tautomer, or a pharmaceutically acceptable salt thereof; and (b) exposing the cell to an effective amount of an antimetabolite such as methotrexate, pemetrexed, cytarabine or nelarabine or 5-fluorouracil or capecitabine or their derivatives.

In another embodiment, the method includes inhibiting the growth of a cancer or tumor cell comprising the steps of: (a) contacting the cell with an effective amount of a compound of -(2,4-dihydroxy-5-isopropyl-phenyl)-4-(1-methyl-indol-5-yl)-5-hydroxy-[1,2,4]triazole, or a tautomer, or a pharmaceutically acceptable salt thereof; and (b) exposing the cell to an effective amount of cytarabine.

In another embodiment, the method includes inhibiting the growth of a cancer or tumor cell comprising the steps of: (a) contacting the cell with an effective amount of a compound of -(2,4-dihydroxy-5-isopropyl-phenyl)-4-(1-methyl-indol-5-yl)-5-hydroxy-[1,2,4]triazole, or a tautomer, or a pharmaceutically acceptable salt thereof; and (b) exposing the cell to an effective amount of nelarabine.

In another embodiment, the method includes inhibiting the growth of a cancer or tumor cell comprising the steps of: (a) contacting the cell with an effective amount of a compound of -(2,4-dihydroxy-5-isopropyl-phenyl)-4-(1-methyl-indol-5-yl)-5-hydroxy-[1,2,4]triazole, or a tautomer, or a pharmaceutically acceptable salt thereof; and (b) exposing the cell to an effective amount of 5-fluorouracil.

In another embodiment, the method includes inhibiting the growth of a cancer or tumor cell comprising the steps of: (a) contacting the cell with an effective amount of a compound of -(2,4-dihydroxy-5-isopropyl-phenyl)-4-(1-methyl-indol-5-yl)-5-hydroxy-[1,2,4]triazole, or a tautomer, or a pharmaceutically acceptable salt thereof; and (b) exposing the cell to an effective amount of capecitabine.

In another embodiment, the method includes inhibiting the growth of a cancer or tumor cell comprising the steps of: (a) contacting the cell with an effective amount of a compound of -(2,4-dihydroxy-5-isopropyl-phenyl)-4-(1-methyl-indol-5-yl)-5-hydroxy-[1,2,4]triazole, or a tautomer, or a pharmaceutically acceptable salt thereof; and (b) exposing the cell to an effective amount of methotrexate.

In another embodiment, the method includes inhibiting the growth of a cancer or tumor cell comprising the steps of: (a) contacting the cell with an effective amount of a compound of -(2,4-dihydroxy-5-isopropyl-phenyl)-4-(1-methyl-indol-5-yl)-5-hydroxy-[1,2,4]triazole, or a tautomer, or a pharmaceutically acceptable salt thereof; and (b) exposing the cell to an effective amount of pemetrexed.

In another embodiment, the method includes inhibiting the growth of a cancer or tumor cell comprising the steps of: (a) contacting the cell with an effective amount of a compound of 5-hydroxy-4-(5-hydroxy-4-(1-methyl-1H-indol-5-yl)-4H-1,2,4-triazol-3-yl)-2-isopropylphenyl dihydrogen phosphate, or tautomer or a pharmaceutically acceptable salt thereof; and (b) exposing the cell to an effective amount of an antimetabolite such as methotrexate, pemetrexed, cytarabine or nelarabine or 5-fluorouracil or capecitabine or their derivatives.

In another embodiment, the method includes inhibiting the growth of a cancer or tumor cell comprising the steps of: (a) contacting the cell with an effective amount of a compound of 5-hydroxy-4-(5-hydroxy-4-(1-methyl-1H-indol-5-yl)-4H-1,2,4-triazol-3-yl)-2-isopropylphenyl dihydrogen phosphate, or tautomer or a pharmaceutically acceptable salt thereof; and (b) exposing the cell to an effective amount of cytarabine.

In another embodiment, the method includes inhibiting the growth of a cancer or tumor cell comprising the steps of: (a) contacting the cell with an effective amount of a compound of 5-hydroxy-4-(5-hydroxy-4-(1-methyl-1H-indol-5-yl)-4H-1,2,4-triazol-3-yl)-2-isopropylphenyl dihydrogen phosphate, or tautomer or a pharmaceutically acceptable salt thereof; and (b) exposing the cell to an effective amount of nelarabine.

In another embodiment, the method includes inhibiting the growth of a cancer or tumor cell comprising the steps of: (a) contacting the cell with an effective amount of a compound of 5-hydroxy-4-(5-hydroxy-4-(1-methyl-1H-indol-5-yl)-4H-1,2,4-triazol-3-yl)-2-isopropylphenyl dihydrogen phosphate, or tautomer or a pharmaceutically acceptable salt thereof; and (b) exposing the cell to an effective amount of 5-fluorouracil.

In another embodiment, the method includes inhibiting the growth of a cancer or tumor cell comprising the steps of: (a) contacting the cell with an effective amount of a compound of 5-hydroxy-4-(5-hydroxy-4-(1-methyl-1H-indol-5-yl)-4H-1,2,4-triazol-3-yl)-2-isopropylphenyl dihydrogen phosphate, or tautomer or a pharmaceutically acceptable salt thereof; and (b) exposing the cell to an effective amount of capecitabine.

In another embodiment, the method includes inhibiting the growth of a cancer or tumor cell comprising the steps of: (a) contacting the cell with an effective amount of a compound of 5-hydroxy-4-(5-hydroxy-4-(1-methyl-1H-indol-5-yl)-4H-1,2,4-triazol-3-yl)-2-isopropylphenyl dihydrogen phosphate, or tautomer or a pharmaceutically acceptable salt thereof; and (b) exposing the cell to an effective amount of methotrexate.

In another embodiment, the method includes inhibiting the growth of a cancer or tumor cell comprising the steps of: (a) contacting the cell with an effective amount of a compound of 5-hydroxy-4-(5-hydroxy-4-(1-methyl-1H-indol-5-yl)-4H-1,2,4-triazol-3-yl)-2-isopropylphenyl dihydrogen phosphate, or tautomer or a pharmaceutically acceptable salt thereof; and (b) exposing the cell to an effective amount of pemetrexed.

In general, the recommended daily dose range of the triazolone compound of formulae (I) or (Ia) for the conditions described herein lie within the range of from about 0.01 mg to about 1000 mg per day, given as a single once-a-day dose preferably as divided doses throughout a day. In an embodiment, the daily dose is administered twice daily in equally divided doses. Specifically, a daily dose range should be from about 5 mg to about 500 mg per day, more specifically, between about 10 mg and about 200 mg per day. In managing the patient, the therapy should be initiated at a lower dose, perhaps about 1 mg to about 25 mg, and increased if necessary up to about 200 mg to about 1000 mg per day as either a single dose or divided doses, depending on the patient's global response. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with individual patient response.

Different therapeutically effective amounts may be applicable for different cancers, as will be readily known by those of ordinary skill in the art. Similarly, amounts sufficient to prevent, manage, treat or ameliorate such cancers, but insufficient to cause, or sufficient to reduce, adverse effects associated with the triazolone compound of formulae (I) or (Ia) described herein are also encompassed by the above described dosage amounts and dose frequency schedules. Further, when a patient is administered multiple dosages of the triazolone compound of formulae (I) or (Ia) described herein, not all of the dosages need be the same. For example, the dosage administered to the patient may be increased to improve the prophylactic or therapeutic effect of the compound or it may be decreased to reduce one or more side effects that a particular patient is experiencing.

In a specific embodiment, the dosage of the composition comprising the triazolone compound of formulae (I) or (Ia) described herein administered to prevent, treat, manage, or ameliorate cancer, or one or more symptoms thereof in a patient is 150 μg/kg, preferably 250 μg/kg, 500 μg/kg, 1 mg/kg, 5 mg/kg, 10 mg/kg, 25 mg/kg, 50 mg/kg, 75 mg/kg, 100 mg/kg, 125 mg/kg, 150 mg/kg, or 200 mg/kg or more of a patient's body weight. In another embodiment, the dosage of the composition comprising a compound described herein administered to prevent, treat, manage, or ameliorate cancer, or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg. The unit dose can be administered 1, 2, 3, 4 or more times daily, or once every 2, 3, 4, 5, 6 or 7 days, or once weekly, once every two weeks, once every three weeks or once monthly.

In certain embodiments, when the triazolone compound of formulae (I) or (Ia) described herein are administered in combination with an antimetabolite, the therapies are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In an embodiment, two or more therapies are administered within the same patient visit.

In certain embodiments, the Hsp90 inhibitor and the antimetabolite described herein and one or more other the therapies (e.g., therapeutic agents) are cyclically administered. Cycling therapy involves the administration of a first therapy (e.g., a first prophylactic or therapeutic agents) for a period of time, followed by the administration of a second therapy (e.g., a second prophylactic or therapeutic agents) for a period of time, followed by the administration of a third therapy (e.g., a third prophylactic or therapeutic agents) for a period of time and so forth, and repeating this sequential administration, i.e., the cycle in order to reduce the development of resistance to one of the agents, to avoid or reduce the side effects of one of the agents, and/or to improve the efficacy of the treatment.

In certain embodiments, administration of the same compound described herein may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In a specific embodiment, a method of preventing, treating, managing, or ameliorating cancer, or one or more symptoms thereof, the methods comprising administering to a subject in need thereof a dose of at least 150 μg/kg, preferably at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds described herein once every day, preferably, once every 2 days, once every 3 days, once every 4 days, once every 5 days, once every 6 days, once every 7 days, once every 8 days, once every 10 days, once every two weeks, once every three weeks, or once a month. Alternatively, the dose can be divided into portions (typically equal portions) administered two, three, four or more times a day.

The invention also provides the use of a compound of formulae (I) or (Ia) or a pharmaceutically acceptable salt thereof for the manufacture of a medicament for the treatment of a subject with cancer. The invention further provides the use of a compound of formulae (I) or (Ia) or a pharmaceutically acceptable salt thereof for the manufacture of a medicament for the treatment of a subject with a cancer in combination with one or more of antimetabolites such as methotrexate, pemetrexed, 5-fluorouracil, 5-fluorouracil prodrugs such as capecitabine, 5-fluorodeoxyuridine monophosphate, cytarabine, cytarabine prodrugs such as nelarabine, 5-azacytidine, gemcitabine, mercaptopurine, thioguanine, azathioprine, adenosine, pentostatin, erythrohydroxynonyladenine, and cladribine.

The invention also provides the use of a compound of formulae (I) or (Ia) or a pharmaceutically acceptable salt thereof for the manufacture of a medicament for the treatment of a subject with NSCLC cancer. The invention further provides the use of a compound of formulae (I) or (Ia) or a pharmaceutically acceptable salt thereof for the manufacture of a medicament for the treatment of a subject with NSCLC in combination with one or more of antimetabolites such as methotrexate, pemetrexed, 5-fluorouracil, 5-fluorouracil prodrugs such as capecitabine, 5-fluorodeoxyuridine monophosphate, cytarabine, cytarabine prodrugs such as nelarabine, 5-azacytidine, gemcitabine, mercaptopurine, thioguanine, azathioprine, adenosine, pentostatin, erythrohydroxynonyladenine, and cladribine.

The invention also provides the use of a compound of formulae (I) or (Ia) or a pharmaceutically acceptable salt thereof for the manufacture of a medicament for the treatment of a subject with NSCLC with a KRAS mutation. The invention further provides the use of a compound of formulae (I) or (Ia) or a pharmaceutically acceptable salt thereof for the manufacture of a medicament for the treatment of a subject with NSCLC with a KRAS mutation in combination with one or more of antimetabolites such as methotrexate, pemetrexed, 5-fluorouracil, 5-fluorouracil prodrugs such as capecitabine, 5-fluorodeoxyuridine monophosphate, cytarabine, cytarabine prodrugs such as nelarabine, 5-azacytidine, gemcitabine, mercaptopurine, thioguanine, azathioprine, adenosine, pentostatin, erythrohydroxynonyladenine, and cladribine.

The invention also provides a compound of formulae (I) or (Ia) or a pharmaceutically acceptable salt thereof for use in treating a subject with a cancer. The invention also provides a compound of formulae (I) or (Ia) or a pharmaceutically acceptable salt thereof for use in treating a subject with cancer in combination with one or more of antimetabolites such as methotrexate, pemetrexed, 5-fluorouracil, 5-fluorouracil prodrugs such as capecitabine, 5-fluorodeoxyuridine monophosphate, cytarabine, cytarabine prodrugs such as nelarabine, 5-azacytidine, gemcitabine, mercaptopurine, thioguanine, azathioprine, adenosine, pentostatin, erythrohydroxynonyladenine, and cladribine.

The invention also provides a compound of formulae (I) or (Ia) or a pharmaceutically acceptable salt thereof for use in treating a subject with NSCLC. The invention also provides a compound of formulae (I) or (Ia) or a pharmaceutically acceptable salt thereof for use in treating a subject with NSCLC in combination with one or more of antimetabolites such as methotrexate, pemetrexed, 5-fluorouracil, 5-fluorouracil prodrugs such as capecitabine, 5-fluorodeoxyuridine monophosphate, cytarabine, cytarabine prodrugs such as nelarabine, 5-azacytidine, gemcitabine, mercaptopurine, thioguanine, azathioprine, adenosine, pentostatin, erythrohydroxynonyladenine, and cladribine.

The invention also provides a compound of formulae (I) or (Ia) or a pharmaceutically acceptable salt thereof for use in treating a subject with NSCLC with a KRAS mutation. The invention also provides a compound of formulae (I) or (Ia) or a pharmaceutically acceptable salt thereof for use in treating a subject with NSCLC with a KRAS mutation in combination with one or more of antimetabolites such as methotrexate, pemetrexed, 5-fluorouracil, 5-fluorouracil prodrugs such as capecitabine, 5-fluorodeoxyuridine monophosphate, cytarabine, cytarabine prodrugs such as nelarabine, 5-azacytidine, gemcitabine, mercaptopurine, thioguanine, azathioprine, adenosine, pentostatin, erythrohydroxynonyladenine, and cladribine.

### EXAMPLES

### Example 1

### In Vitro Combination Analysis of Ganetespib with Cytarabine (AraC) and Nelarabine (AraG)

### A. Materials and Methods

Cell Lines

Human MOLT-3, MOLT-4 and Jurkat T cell leukemia cells were purchased from the American Type Culture Collection (Manassas, Va.) and grown in RPMI (Sigma), following ATCC recommendations, in the presence of fetal bovine serum (10%), 2 mM L-glutamine and antibiotics (100 IU/ml penicillin and 100 μg/ml streptomycin, Sigma). Cells were maintained at 37° C., 5% CO₂ atmosphere.

Cell Viability Assays

Cell viability was measured using the alamarBlue assay (Invitrogen). In brief, cells were plated in 96-well plates in triplicate at 20K, 15K or 15K cells per well for MOLT-3, MOLT-4 or Jurkat cells respectively, and incubated at 37° C., 5% CO₂ atmosphere for 24 hr prior to the addition of drug or vehicle (0.3% DMSO) to the culture medium. After 72 hr, 10 μl/well Alamar Blue was added to the wells and incubated for an additional 3 hr at 37° C., 5% CO₂ atmosphere. Fluorescence (560EX/590EM nM) was measured with a SpectraMax microplate reader (Molecular Devices) and the resulting data were used to calculate cell viability, normalized to vehicle control.

B. Combination Studies of Ganetespib with Ara-C and with Ara-G.

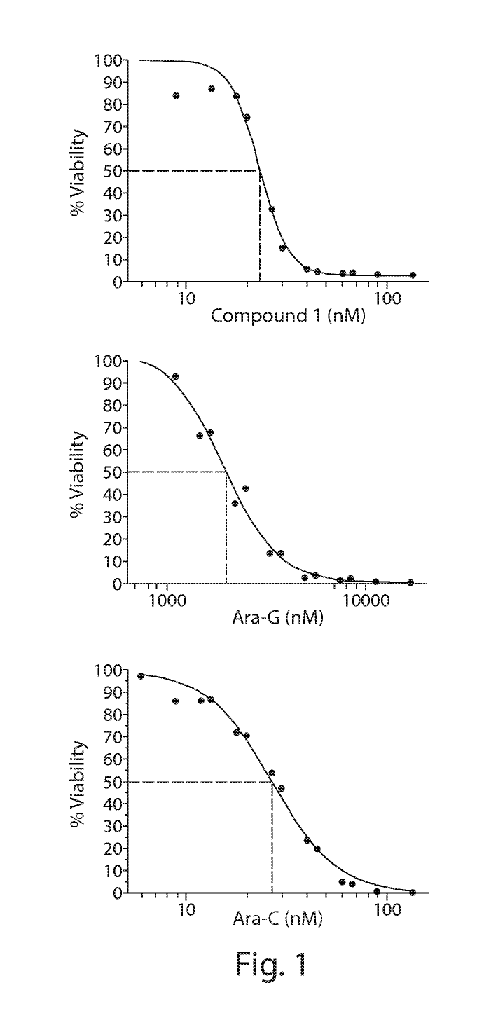

The half maximal inhibitory concentration (IC₅₀) for ganetespib (synthesized at Synta Pharmaceuticals), Ara-C and Ara-G (Sigma) were first determined using a 1.5-fold serial dilution series of compound. After MOLT-4 cells were exposed to drug for 72 hr, cell viability was measured and results were fit to a four parameter logistic model (XLFit, ID Business Solutions) shown in FIG. 1. The IC₅₀ for ganetespib was calculated at approximately 20 nM, 25 nM for Ara-C and 2 μM for Ara-G.

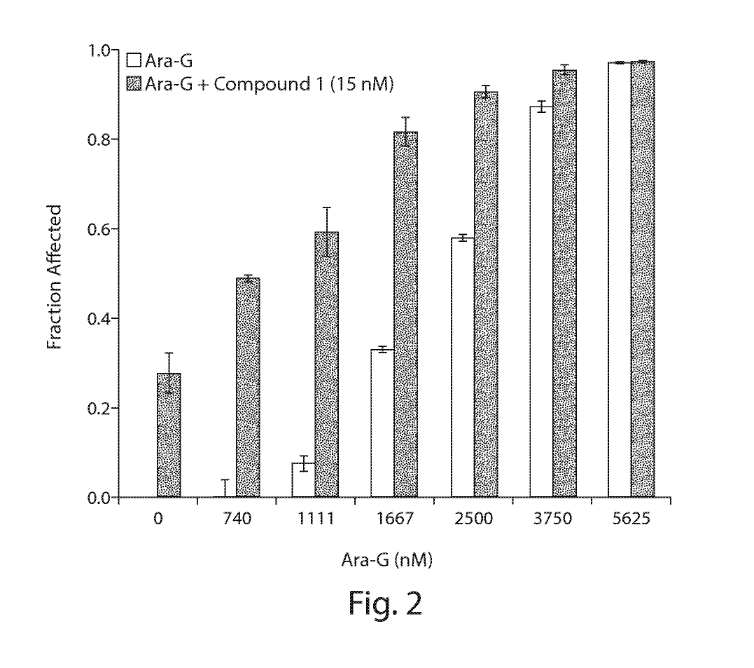

Combinations between ganetespib and the nucleoside analog Ara-G were then performed in MOLT-4 cells concurrently based on the IC₅₀ for each agent. The combined drugs, as well as each drug alone, were incubated with the cells for 3 days and the surviving fraction of cells relative to control was determined using the alamarBlue assay. Shown in FIG. 2, the combination of ganetespib with Ara-G displayed enhanced cytotoxicity relative to either agent alone.

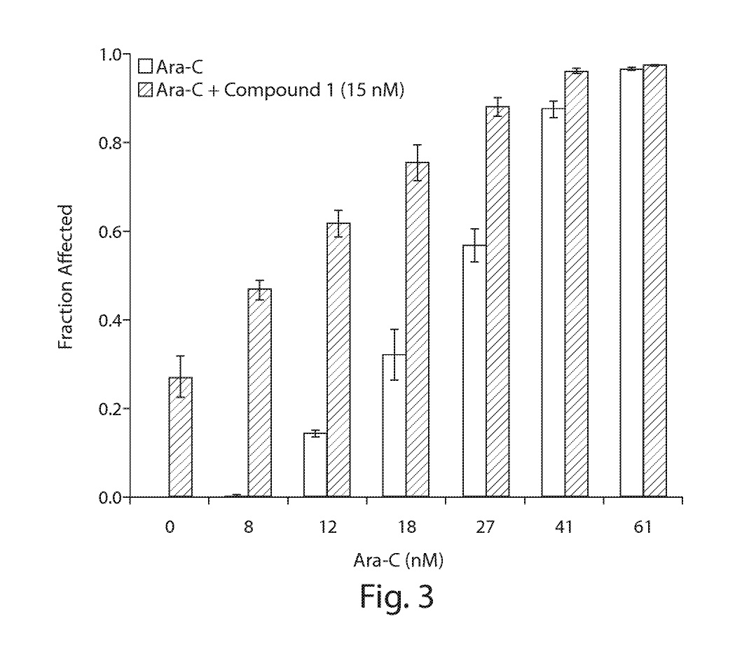

Similar results were observed in MOLT-4 cells with the combination of ganetespib and Ara-C, another nucleoside analog differing from Ara-G in the base moiety (FIG. 3).

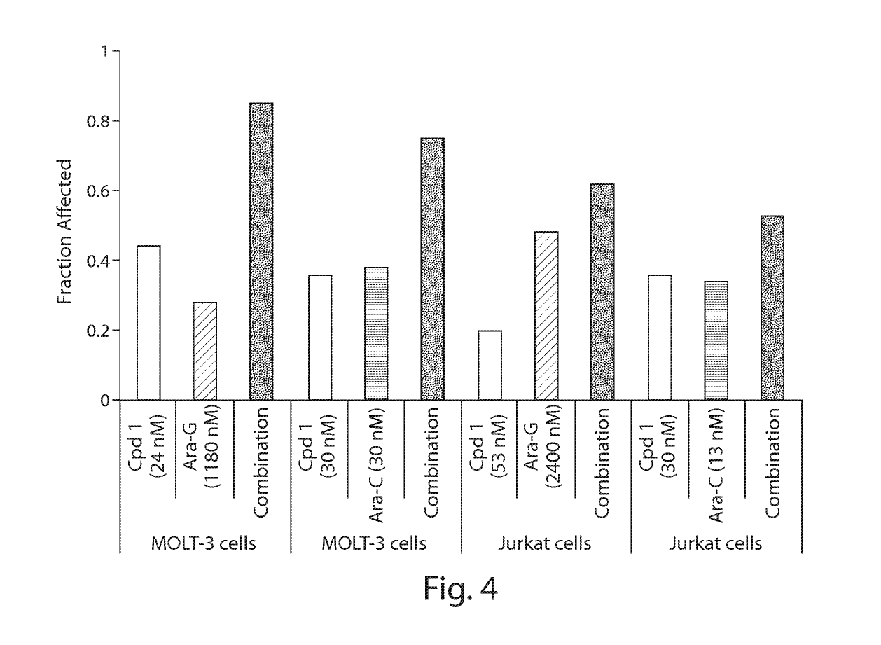

To determine if this was cell type specific, combinations were performed in additional T-cell leukemia cells. As shown in FIG. 4, both Ara-C and Ara-G improved the activity of ganetespib in MOLT-3 and Jurkat cells. Taken together, this data supported the use of ganetespib in combination with the nucleosides Ara-C and Ara-G in T-cell leukemia.

### Example 2

### In Vitro Combination Analysis of Ganetespib with Fluorourail in CRC

### A. Materials and Methods

Cell Lines

Human HCT-116 colorectal cancer cells (CRC) were purchased from the American Type Culture Collection (Manassas, Va.) and grown in McCoy's 5a media (Sigma), following ATCC recommendations, in the presence of fetal bovine serum (10%), 2 mM L-glutamine and antibiotics (100 IU/ml penicillin and 100 μg/ml streptomycin, Sigma). Cells were maintained at 37° C., 5% CO₂ atmosphere.

Cell Viability Assays

Cell viability was measured using the alamarBlue assay (Invitrogen). In brief, cells were plated in 96-well plates in triplicate at 5K cells per well and incubated at 37° C., 5% CO₂ atmosphere for 24 hr prior to the addition of drug or vehicle (0.3% DMSO) to the culture medium. After 72 hr, 10 μl/well alamarBlue was added to the wells and incubated for an additional 3 hr at 37° C., 5% CO₂ atmosphere. Fluorescence (560EX/590EM nM) was measured with a SpectraMax microplate reader (Molecular Devices) and the resulting data were used to calculate cell viability, normalized to vehicle control.

B. Combination Studies with Ganetespib and Fluorouracil

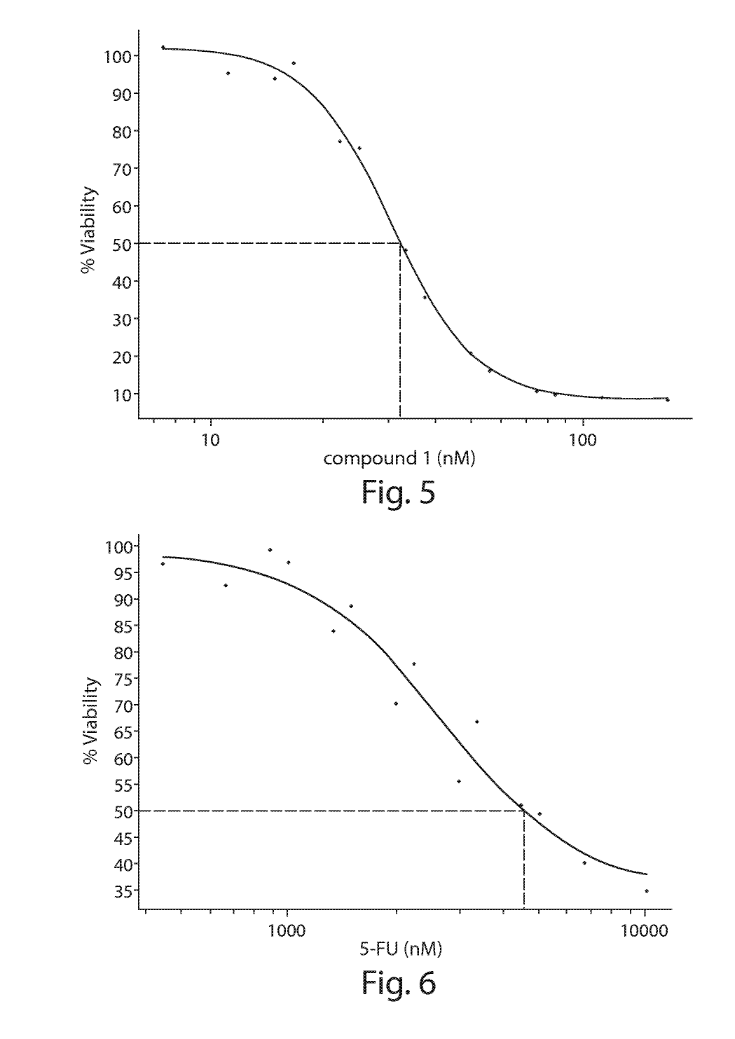

The half maximal inhibitory concentration (IC₅₀) for ganetespib (synthesized at Synta Pharmaceuticals) and fluorouracil (5-FU) (purchased from Sigma) were first determined using a 1.5-fold serial dilution series of compound. After HCT-116 cells were exposed to drug for 72 hr, cell viability was measured and results were fit to a four parameter logistic model (XLFit, ID Business Solutions). The IC₅₀ for ganetespib was calculated at approximately 32 nM, and 4.5 μM for 5-FU (FIGS. 5 and 6).

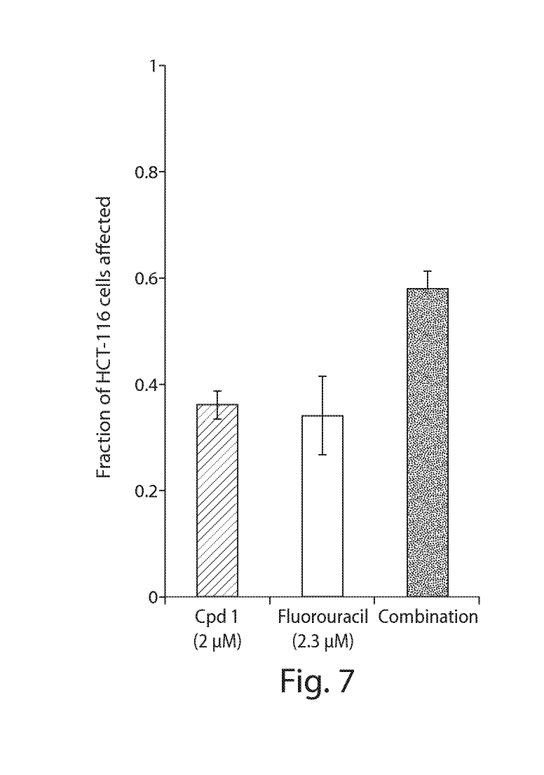

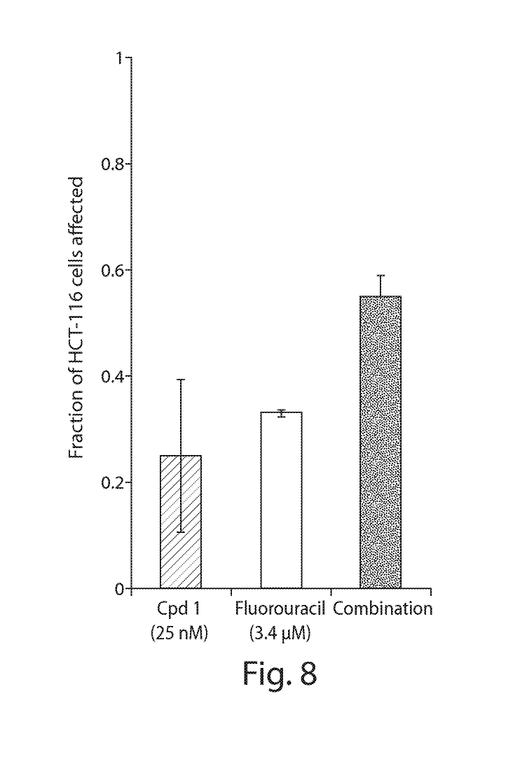

Combinations between ganetespib and fluorouracil were then performed in HCT-116 cells concurrently based on the IC₅₀ for each agent in matrix format with 54 combination pairs for each drug. The combined drugs, as well as each drug alone, were incubated with the cells for 3 days and the surviving fraction of cells relative to control was determined using the alamarBlue assay. Combination results are shown in FIGS. 7 and 8. The combination of ganetespib with 5-FU displayed enhanced cytotoxicity relative to single agent drugs alone. Similar results were observed when cells were exposed to ganetespib for just one hour, washed and then treated with fluorouracil for 3 days. Taken together, this data supports the use of ganetespib in combination with fluorouracil in solid cancers including gastric, bladder and colorectal.

### Example 3

### Ganetespib in Combination with Standard of Care Chemotherapies Displays Efficacy in NSCLC Cancer Subtypes with KRAS Mutations

Mutant KRAS is detected in 20-25% of non-small cell lung carcinomas (NSCLC) and represents one of the most common oncogenic drivers of this disease. NSCLC tumors with oncogenic KRAS respond poorly to currently available therapies necessitating the pursuit of new treatment strategies. Recent results from a Phase 2 trial with ganetespib revealed that >60% of patients with NSCLC having a KRAS mutation exhibited tumor shrinkage at 8 weeks, indicating that ganetespib is useful in the treatment of this disease.

To further understand the actions of ganetespib in NSCLC tumors having a KRAS mutation, studies were executed in a diverse panel of KRAS mutant NSCLC cell lines to investigate whether ganetespib is effective in suppressing critical cell signaling nodes responsible for KRAS-driven NSCLC cell survival and to assess whether ganetespib can synergize with both clinical agents targeted against these signaling nodes and standard of care chemotherapies.

For combinatorial analysis, cells were seeded in 96-well plates at a predetermined, optimum growth density for 24 h prior to the addition of drug or vehicle to the culture medium. Drug combinations were applied at a non-constant ratio over a range of concentrations for 72 or 96 hours. For each compound tested, a 7 point dose range was generated based on 1.5 fold serial dilutions using IC₅₀ values set as the mid-point. Cell viability was assessed by either AlamarBlue® (Invitrogen, Carlsbad, Calif.) or CellTiter-Glo® assays and normalized to vehicle controls. For each combination study, the level of growth inhibition (fraction affected) is plotted relative to vehicle control. Data are presented as one relevant combination point and the corresponding single agent data for each cell line tested.

Ganetespib displayed potent anticancer activity across 15 KRAS mutant NSCLC cell lines assayed in vitro, with an average IC₅₀ of 24 nM. Combining ganetespib with anti-mitotics, alkylating agents or topoisomerase inhibitors resulted in an increase in cell death of up to 44, 61 and 26%, respectively, versus monotherapy. At the molecular level, ganetespib induced the destabilization of several KRAS substrates, including BRAF and CRAF, leading to inactivation of their downstream effectors followed by programmed cell death. Ganetespib effectively suppressed the growth of human KRAS mutant NSCLC tumor xenografts in vivo.

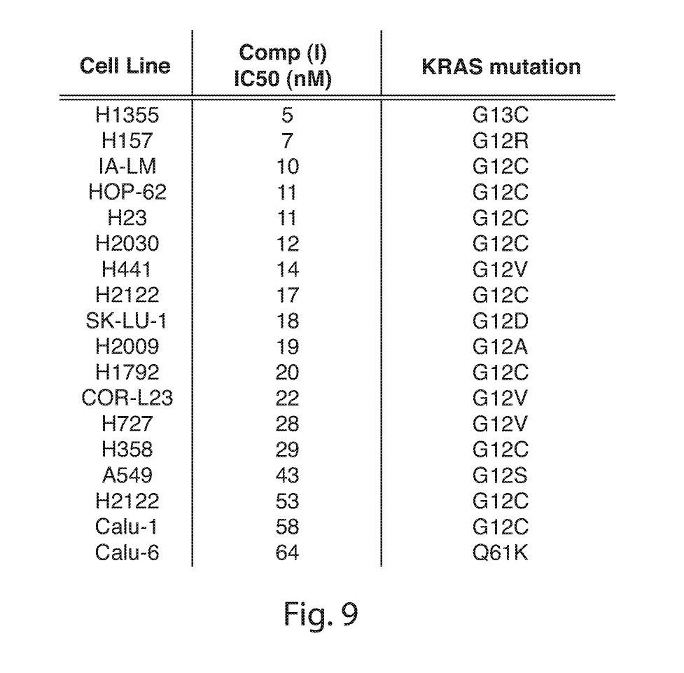

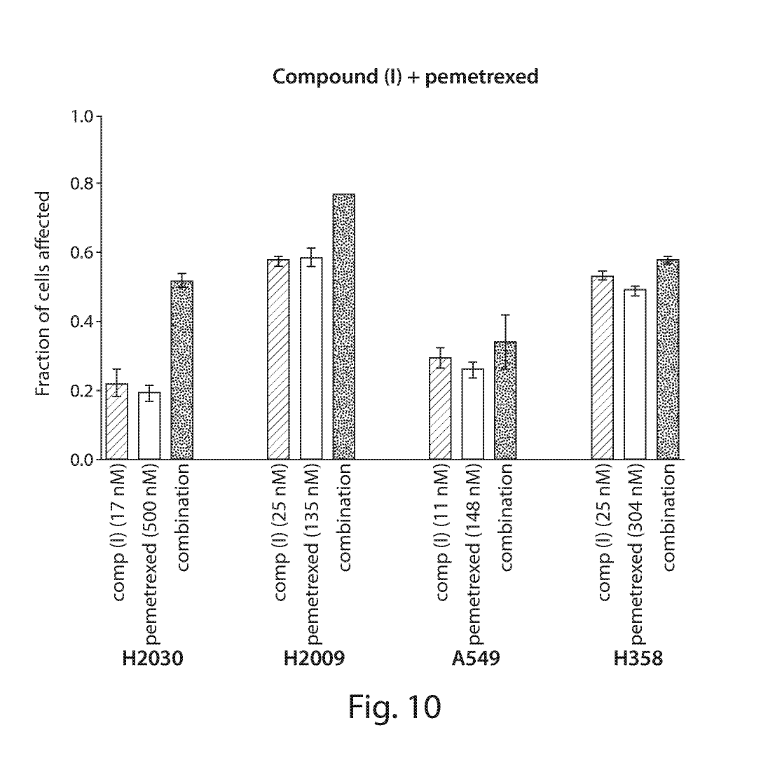

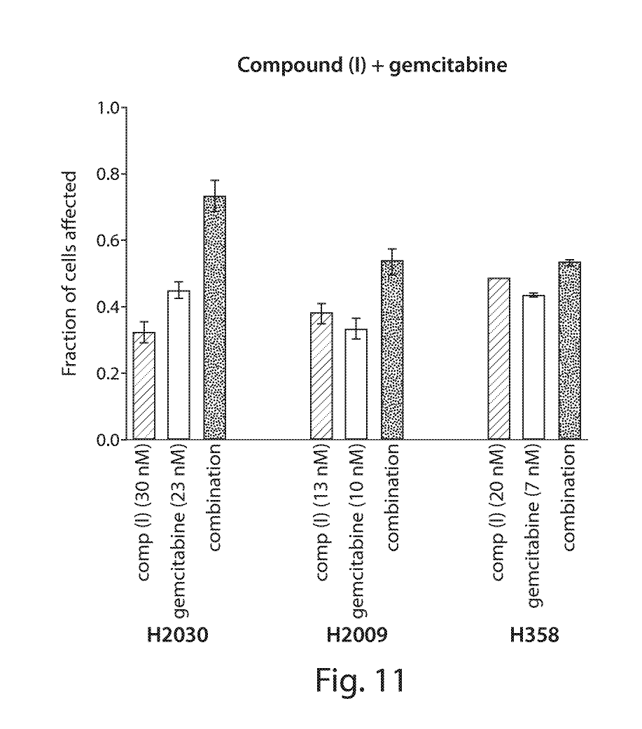

More particularly, ganetespib elicited promising activity against mutant KRAS NSCLC tumor cells (FIG. 9). In order to further identify feasible strategies to enhance the anti-tumor activity of ganetespib, combination studies were performed with standard of care chemotherapies in mutant KRAS NSCLC cell lines. It was found that combining ganetespib with the antimetabolite pemetrexed enhanced cell death by 2.4 and 1.5 fold for H2030 and H2009 cells, respectively, while a marginal increase in cytotoxicity was observed for A549 and H358 cells (FIG. 10). Ganetespib in combination with the nucleoside analog, gemcitabine, increased cell death 2.3 and 1.4 fold for H2009 and A549 cells, respectively, and no benefit was observed for H358 cells (FIG. 11). Standard of care chemotherapeutics utilized in KRAS mutant NSCLC show activity with ganetespib in vitro. Pemetrexed and gemcitabine showed up to 4 fold increases in cell death when combined with ganetespib. None of the agents antagonized the anticancer activity of ganetespib.

In summary, ganetespib, a potent inhibitor of Hsp90, has shown encouraging evidence of clinical activity, including tumor shrinkage in patients with KRAS mutant NSCLC. In vitro, ganetespib exhibited potent anticancer activity in NSCLC cells with a diverse spectrum of KRAS mutations due in part to degradation and inactivation of critical KRAS signaling effectors. Combination with targeted therapies that overlap with these signaling nodes led to enhanced anticancer activity in vitro and in mouse models of KRAS mutant NSCLC. Taken together, these results demonstrate clinical utility of ganetespib in patients with KRAS mutant NSCLC.

All publications, patent applications, patents, and other documents cited herein are incorporated by reference in their entirety. In case of conflict, the present specification, including definitions, will control. In addition, the materials, methods, and examples throughout the specification are illustrative only and not intended to be limiting in any way.

1. A method of treating cancer in a subject, comprising administering to the subject an effective amount of a triazolone compound of formula (I) or (Ia): 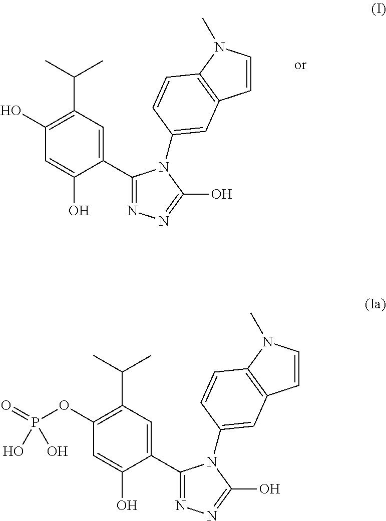 [SMILES: CC(C)c1cc(-c2nnc(O)n2-c2ccc3c(ccn3C)c2)c(O)cc1O ; CC(C)c1cc(-c2nnc(O)n2-c2ccc3c(ccn3C)c2)c(O)cc1OP(=O)(O)O]
or a tautomer, or a pharmaceutically acceptable salt thereof, in combination with an antimetabolite selected from methotrexate, pemetrexed, cytarabine, nelarabine, 5-fluorouracil, and capecitabine, wherein the cancer is leukemia, acute lymphoblastic leukemia, chronic lymphocytic leukemia, acute myelogenous leukemia, chronic myelogenous leukemia, T-cell acute lymphoblastic leukemia, T cell prolymphocytic leukemia, B-cell leukemia, lymphoma, non-Hodgkin's lymphoma, T-cell lymphoblastic lymphoma, a solid cancer, gastric cancer, bladder cancer, non-small cell lung cancer, breast cancer, or colorectal cancer.

2. The method of claim 1, wherein the triazolone compound is according to formula (I), or a tautomer, or a pharmaceutically acceptable salt thereof.

3. The method of claim 1, wherein the antimetabolite is cytarabine.

4. The method of claim 1, wherein the antimetabolite is nelarabine.

5. The method of claim 1, wherein the antimetabolite is 5-fluorouracil.

6. The method of claim 1, wherein the antimetabolite is capecitabine.

7. The method of claim 1, wherein the antimetabolite is methotrexate.

8. The method of claim 1, wherein the antimetabolite is pemetrexed.

9. The method of claim 1, wherein the cancer is leukemia, acute lymphoblastic leukemia, chronic lymphocytic leukemia, acute myelogenous leukemia, chronic myelogenous leukemia, T-cell acute lymphoblastic leukemia, T cell prolymphocytic leukemia, lymphoma, non-Hodgkin's lymphoma, T-cell lymphoblastic lymphoma, a solid cancer, gastric cancer, bladder cancer, non-small cell lung cancer, breast cancer, or colorectal cancer.

10. The method of claim 9, wherein the cancer is T-cell acute lymphoblastic leukemia or T-cell lymphoblastic lymphoma.

11. The method of claim 9, wherein the cancer is non-small cell lung cancer.

12. The method of claim 11, wherein the non-small cell lung cancer has a KRAS mutation.

13. The method of claim 1, wherein the compound of formula (I) is administered intravenously at a dose of from about 100 mg/m² to about 200 mg/m².

14-16. (canceled)

17. The method of claim 1, wherein the compound of formula (I) is administered once or twice weekly.

18. The method of claim 3, wherein cytarabine is administered subcutaneously at a dose of from about 20 mg/m² to about 50 mg/m².

19. The method of claim 18, wherein cytarabine is administered twice a day.

20. The method according to claim 4, wherein nelarabine is administered intravenously at a dose of from about 600 mg/m² to about 2000 mg/m².

21-22. (canceled)

23. The method of claim 6, wherein capecitabine is administered at a dose from about 200 mg/m² to about 3000 mg/m².

24. (canceled)

25. The method of claim 1, wherein the method further comprises administering an additional anticancer therapy.

26. The method of claim 25, wherein the additional anticancer therapy is radiotherapy.
